(19) United States
(12) Patent Application Publication
(10) Pub. No.: US 2020/0054271 A1
(43) Pub. Date: Feb. 20, 2020

(54) COMPUTER-BASED LIVER MODEL

(71) Applicants: MAX-PLANCK-GESELLSCHAFT ZUR FORDERUNG DER WISSENSCHAFTEN E.V., München (DE); Technische Universitat Dresden, Dresden (DE)

(72) Inventors: Marino ZERIAL, Dresden (DE); Yannis KALAIDZIDIS, Dresden (DE); Ivo SBALZARINI, Dresden (DE); Kirstin MEYER, San Francisco, CA (US); Oleksandr OSTRENKO, Dresden (DE); Lutz BRUSCH, Dresden (DE)

(21) Appl. No.: 16/487,062

(22) PCT Filed: Feb. 20, 2018
(86) PCT No.: PCT/EP2018/054119

§ 371 Date: Aug. 19, 2019

(30) Foreign Application Priority Data
Feb. 20, 2017 (EP) 17156923.9

(51) Int. Cl. A61B 5/00; A61B 5/055; A61B 90/20

(52) U.S. Cl. CPC A61B 5/4244; A61B 5/055; A61B 5/004; A61B 5/4848; A61B 5/0071; A61B 5/7239; A61B 90/20

(57) ABSTRACT

A method of predicting bile flow through a liver is described. The method comprises: (a) dividing the axis connecting the central vein and a portal vein of a lobule into zones; (b) measuring secretion of bile by hepatocytes; (c) calculating the transport rate of said bile of (b) through each of the zones defined in (a), using differential equations; (d) providing a three-dimensional representation of the bile canaliculi in said lobule; (e) calculating a correction factor as the ratio between hydraulic radius and geometric radius of said bile canaliculi; and (f) calculating bile transport through (f-i) said three-dimensional representation of (d) using the transport rates determined in step (c) and said correction factor calculated in step (e), by solving the Navier-Stokes equations for said representation of (d); or (f-ii) a porous medium model of said liver lobule using the transport rates determined in step (c).

### RELATED APPLICATIONS

This patent application is the National Phase of International Application No. PCT/EP2018/054119, filed Feb. 20, 2018, which designated the U.S. and that International Application was published under PCT Article 21(2) in English, which claims the benefit of priority to European Patent Application No. 17156923.9, filed Feb. 20, 2017. The entire contents of the foregoing applications are incorporated herein by reference, including all text, tables and drawings.

### BACKGROUND

The bile canaliculi (BC) network of the liver transports bile to the intestine for the digestion of nutritional lipids and clearance of metabolic waste products. Due to the detergent-like properties of bile, cholestasis, the impairment of bile flow, induces hepatocellular damage from extracellular accumulation of bile acids, fibrosis and ultimately cirrhosis under chronic conditions (Balistreri et al., 2005; Boyer, 2013; Coleman et al., 1979; Padda et al., 2011; Trauner et al., 1998). The detoxifying function of the liver renders the organ prone to drug-induced liver injury (DILI) (Giri et al., 2010). DILI therefore represents a prevalent problem of pharmacological drug development.

Current diagnostic techniques for DILI and cholestasis are based on the detection of serum markers and imaging methods such as computer tomography. However, these techniques have low resolution and only provide a limited understanding of the underlying disease etiology.

Biomedical investigations of cholestatic liver disease and pharmacological drug safety assessments therefore require a quantitative understanding of biliary fluid dynamics to study and target the regulation of bile flow. Yet, despite its importance, experimental measurements or computational simulations of intra-hepatic bile flow are currently unavailable.

BC are sub-cellular structures of 0.5-2 μm in diameter. They are formed by the apical membranes of counter- and juxta-posed hepatocytes that collectively build a highly ramified 3D tubular network (Elias, 1949). Bile, which is generated by apical secretion from hepatocytes, flows from the central vein (CV) to the portal vein (PV) area of the liver lobule, where it drains into the common bile duct (Elias, 1949). Blood flows counter current through the sinusoidal endothelial network, from PV to CV. Along this porto-central axis, the biliary network displays geometrical and functional differences, including BC branching frequency, diameter and hepatocyte bile acid transport activity (Baumgartner et al., 1986, 1987; Layden and Boyer, 1978; Morales-Navarrete et al., 2015).

### SUMMARY

Due to these heterogeneities, the measurement and simulation of intra-hepatic biliary fluid dynamics require a quantitative approach that considers spatial differences and that bridges the micron (sub-cellular) to the millimeter (lobule) scale. In view of such need, the technical problem underlying the present invention can be seen in the provision of a computer-based model of biliary fluid dynamics in the liver lobule.

This technical problem is solved by the subject-matter of the claims.

The present invention relates to a computer-based method of predicting bile flow through a lobule of a mammalian liver, said method comprising: (a) dividing the axis connecting the central vein and a portal vein of said lobule into a central zone, a middle zone and a portal zone, preferably based on the positions of said central vein and said portal vein as determined from microscopic images; (b) measuring experimentally secretion of bile by hepatocytes; (c) calculating the transport rate of said bile of (b) through each of the zones defined in (a), preferably using ordinary differential equations; (d) providing a three-dimensional representation of the bile canaliculi in said lobule; (e) calculating a first correction factor as the ratio between hydraulic radius and geometric radius of said bile canaliculi; and (f) calculating bile transport through (f-i) said three-dimensional representation of (d) using the transport rates determined in step (c) and said first correction factor calculated in step (e), preferably by solving the Navier-Stokes equations for said three-dimensional representation of (d); or (f-ii) a three-dimensional porous medium model of said liver lobule using the transport rates determined in step (c); thereby predicting said bile flow.

### BRIEF DESCRIPTION OF THE DRAWINGS

FIG. 1 illustrates a 3D multi-scale model strategy.

FIG. 2A illustrates transport through a liver lobule.

FIG. 2B illustrates identifying an axis of a liver lobule.

FIG. 2C illustrates cell layers.

FIG. 2D illustrates clearing CF from different zones (cytoplasm).

FIG. 2E illustrates clearing CF from different zones (bile canaliculi).

FIG. 2F illustrates estimating bile transport rates.

FIG. 2G illustrates data showing how hepatic CF transport differs significantly within the liver lobule.

FIG. 3A illustrates imaged liver tissue.

FIG. 3B illustrates a 3D network reconstruction.

FIG. 3C illustrates porosity data.

FIG. 3D illustrates automated image acquisition of tissue at nanometer resolution.

FIG. 3E illustrates 3D reconstruction of microvilli.

FIG. 4A illustrates simulations of bile flow in a reconstructed BC.

FIG. 4B illustrates bile flow.

FIG. 4C illustrates bile velocity and BC network water influx profiles.

FIG. 5A illustrates visualization of contractile events occurring within a millisecond time frame.

FIG. 5B illustrates a dilation of BC in Fasudil-treated mice.

FIG. 5C illustrates a quantification of BC radius.

FIG. 5D illustrates imaging of CF transport in Fasudil-treated mice.

FIG. 5E illustrates bile velocity plotted against distance from CV.

FIG. 5F illustrates bile velocity/pressure plotted against distance from CV.

FIG. 6A illustrates a prediction of one of two inversely related gradients of bile velocity and pressure within the lobule.

FIG. 6B illustrates a prediction of another one of two inversely related gradients of bile velocity and pressure within the lobule.

FIG. 7A illustrates dilation of BC in a first zone.

FIG. 7B illustrates dilation of BC in a second zone.

FIG. 7C illustrates images related to measured bile velocity by IVM of CF(DA) transport in control mice.

FIG. 7D illustrates data related to measured bile velocity by IVM of CF(DA) transport in control mice.

### DETAILED DESCRIPTION

Bile is one of the principal products of the liver. It is stored in the gall bladder. During digestion, the bile is secreted into the duodenum. Bile is considered to help digestion of lipids because it has emulsifying properties.

At a mesoscopic level, lobules can be seen as the building blocks of the mammalian liver. Lobules have to be held distinct from lobes. A mammalian liver typically comprises a one-digit number of macroscopic lobes, e.g. two major and two minor lobes in humans, and five lobes in mice. At the same time, it comprises approximately between 1 and 1.5 million lobules. Each lobule has an approximately hexagonal cross section and a diameter of 1 to 2 mm in humans. The lobules are formed by multiple cell types, the most abundant by number and mass being the hepatocytes. In the center of each lobule, there is a central vein. At the periphery of each lobule, there are vessels which originate in the portal vein. For the sake of simplicity, said vessels originating from the portal vein and being located at the periphery of each lobule are also referred to as “portal vein” in this specification.

While blood flows from portal veins to the central vein, bile flows in the opposite direction. While blood flows through the mentioned blood vessels, bile flows through a separate system of capillaries, also referred to as bile canaliculi. While vessels and flow direction of bile and blood, respectively, are opposite, the coordinate system in either case is defined by the location of the central vein and a portal vein at the periphery of the lobule.

The present inventors recognized that tissue properties and in particular bile flow properties are not uniform along the coordinate axis defined by central vein and a portal vein. This is accounted for by partitioning in accordance with step (a) of the method in accordance with the first aspect of the present invention, and, as disclosed further below, by step (c).

In a preferred embodiment, the central zone corresponds to approximately one cell layer, the middle zone to approximately six cell layers and the portal zone to approximately three cell layers.

The present invention is not confined to generic modeling of lobule function. Instead, the computer-based method of the present invention allows to be fed with species-specific or even patient-specific parameters and data.

Accordingly, secretion of bile and the amount of secreted bile is to be determined experimentally in accordance with step (b) of the method of the first aspect.

Step (c) provides for calculating the transport rate of bile. A preferred mathematical model used for calculating is a set of ordinary differential equations, wherein said set of ordinary differential equations accounts for the partitioning of the lobule in accordance with step (a).

The processes and interactions described here in terms of ordinary differential equations can alternatively be represented in different mathematical or computational frameworks, especially as agent-based model, colored Petri net, cellular automaton or discrete dynamical system. Such alternative mathematical or computational models can be used as long as they comprise the same processes and interactions described here in terms of ordinary differential equations. The use of such alternative mathematical or computational models will be considered by a person skilled in the art.

More specifically, each of the differential equations defines the derivative with respect to time of the concentration of a tracer or of bile in a given compartment. Preferably, compartments are: cytoplasmic compartment and bile canaliculi compartment. Cytoplasmic compartment and bile canaliculi compartment in turn are further subdivided in accordance with the partitioning defined in step (a), i.e. into a central zone, a middle zone and a portal zone. Particularly preferred implementations are subject of preferred embodiments disclosed further below and described in more detail in section “mathematical 3-compartment model of CF(DA) transport in the liver” of Example 2, respectively.

A hallmark of the present invention is the use of a precise description of the network of the bile canaliculi as well as of how flow of bile occurs within said network. To this end, and in accordance with step (d), a three-dimensional representation of the bile canaliculi is provided. In line with the above mentioned notion of patient-specific modeling, said three-dimensional representation may be determined for a specific individual and subsequently fed into the computer-based method of predicting bile flow of the invention at step (d).

A further feature which has an important influence on the flow characteristics of the bile through the network of canaliculi is the nature of the internal surface of the canaliculi. It turns out that the lumens of the bile canaliculi are convoluted and densely packed with microvilli. This has an important influence on the flow characteristics of any liquid flowing through the canaliculi. This is accounted for by step (e).

Step (f) provides for combining the information about geometrical and hydraulic properties in accordance with steps (d) and (e) with the results of the preceding steps (a) to (c). Depending on the specific question to be addressed by the method in accordance with the present invention, the more involved approach of step (f-i) may be chosen, which is preferred, or a three-dimensional porous medium model of the liver lobule may be used as an approximate representation (f-ii).

An exemplary implementation of (f-ii) is given in the section entitled “A 3D anisotropic porous medium model of biliary fluid dynamics” of Example 2. This comprises determining the porosity along the central vein-portal vein axis.

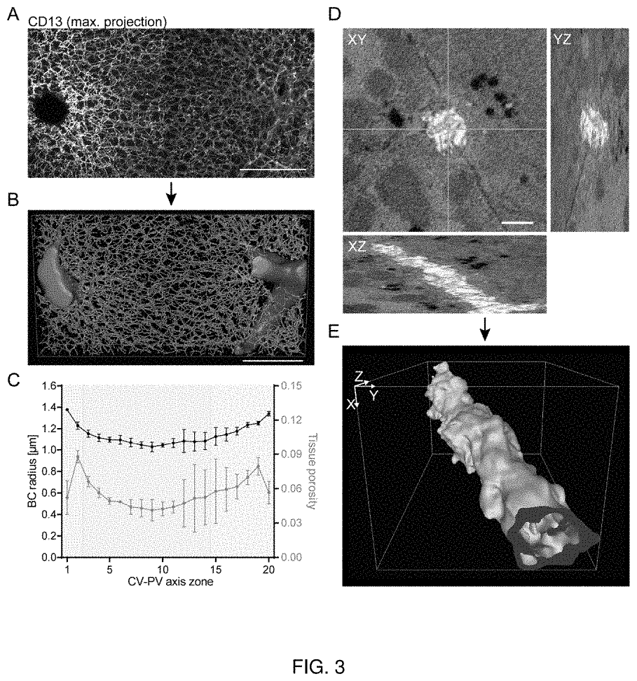

As exemplified by the data in FIG. 3C (grey, right vertical axis), porosity can be quantified by staining the apical membrane, segmenting the images and calculating the CV/PV-position-dependent volume fraction of the bile canaliculi.

BC-porosity can also be determining by measuring sinusoid volume density and using a conversion factor or profile of apical versus basal membrane area per hepatocyte.

For human patients, this information can be extracted from non-invasive Raman microscopy images, followed by image de-noising and segmentation.

In a preferred embodiment, the Navier-Stokes equations are solved numerically. In a further preferred embodiment, bile is considered as an incompressible Newtonian fluid.

The processes and interactions described here in terms of partial differential equations can alternatively be represented in different mathematical or computational frameworks, especially as Lattice-Boltzmann equations, particle discretisation methods or lattice-gas cellular automata. Such alternative mathematical or computational models can be used as long as they comprise the same processes and interactions described here in terms of the Navier-Stokes equation. The use of such alternative mathematical or computational models will be considered by a person skilled in the art.

In a further preferred embodiment, said first correction factor is in the range between 0.2 and 0.5, more preferably between 0.3 and 0.4. In another preferred embodiment, it is individually determined for a given disease state or individual.

With the first aspect, the inventors provide a predictive 3D multi-scale model that simulates fluid dynamic properties successively from the sub-cellular to the tissue level. As will become apparent from preferred embodiments disclosed further below, the model in accordance with the first aspect integrates structural and functional properties of the mammalian liver lobule which are preferably determined by high resolution confocal intravital microscopy, serial block-phase scanning electron microscopy, and image analysis techniques (see Example 4).

As noted above, the method permits calculating a spatial profile of velocity and pressure. Surprisingly, and as can be seen from the detailed description further below and the enclosed examples, there is significant spatial heterogeneity of biliary geometry and hepatocytes transport activity which in turn establish gradients of bile velocity and pressure within the liver lobule (see Example 4).

Owing to its sound basis on experimental data, in particular the three-dimensional representation of the bile canaliculi in accordance with step (d), the predictive performance is good. In the proof of principle study, the response of the liver lobule to acetaminophen has been predicted (see Example 5) which prediction is in agreement with experimental measurements. More generally speaking, the method in accordance with the first aspect is a tool which allows to functionally characterize liver diseases and quantitatively estimate biliary transport upon drug-induced liver injury (DILI).

While being solidly based on experimental data, it is noteworthy that the model of the invention, i.e. the method in accordance with the first aspect, does not require to determine experimentally each and every parameter. Instead, it is the evolved nature of the model which renders certain measurements dispensable. In particular, it is not necessary to measure parameters which are difficult to measure such as osmotic pressure, the water permeability of the hepatocyte membrane or the local bile viscosity. Instead, it is preferred to determine experimentally only: (i) bile canaliculi diameter distribution along the CV-PV axis, (ii) the integral bile flux between zones, and (iii) the mesoscopic characterization of the network of bile canaliculi.

The bile canaliculi diameter distribution of (i) is obtained by light microscopy, preferably high resolution fluorescence microscopy. Preferably, electron microscopy is furthermore used to determine the correction factor of step (e) of the method of the first aspect. Based on these measurements, the property a(x) as used in equation (IV) (see further below) can be determined.

The term “intergral bile flux” refers to bile flux in bile canaliculi and cytoplasm of hepatocytes. Bile flux occurs in the above-defined three zones (central zone, middle zone and portal zone). Intergral bile flux of (ii) is determined by intravital microscopy or non-invasive imaging methods such as Raman microscopy or micro-MRI as described herein. To render flux detectable, preferably tracer molecules such as fluorescent tracers are used. Intergral bile flux enters the model of the first aspect at step (c) and preferred implementations thereof disclosed further below; see equations (Ia) to (Ic), (IIa) to (IIc) and (IIIa) to (IIIc).

Experimental data for said mesoscopic characterization of the network of bile canaliculi of (iii) is obtained by confocal microscopy and enters the model at step (d) of the method of the first aspect. Electron microscopy could also be used for this purpose.

Said three parameters (i), (ii) and (iii) can also be determined in the diseased state or upon administration of an agent or drug, preferred agents and drugs being disclosed further below. This in turn allows modeling of a diseased state or the state of a liver lobule in response to an agent or drug.

In a preferred embodiment, the method further comprises calculating (g) a spatial profile of bile flow velocity and/or pressure in said canaliculi as a function of the coordinate along said axis defined in step (a) and based on the bile transport calculated in step (f); and/or (h) a second correction factor accounting for a peristaltic component of bile flow.

Step (g) provides for calculating bile flow velocity and pressure in the canaliculi as a function of the coordinate which has been defined in step (a) by connecting the positions of the central vein with that of a portal vein. It is noted that step (c) permits to calculate the total bile flux in the lobule. It does not provide information, however, about the distribution of flux within the lobule. The knowledge of the distribution of flux, however, is key for accurate prediction. The latter is only possible by fully taking into account the bile canaliculi diameter which changes within the lobule, in particular allowing the coordinate defined in step (a), and furthermore in response to perturbations. In other words, the steps following step (c) provide for an integration of tissue morphology and bile flux within the same model or method of predicting bile flow.

As regards step (h), it is noted that in the prior art, there were speculations about whether the flow of bile is merely osmotically driven, or whether there is a peristaltic contribution. In order to address this unresolved problem, the method in accordance with the above disclosed preferred embodiment, accounts for a peristaltic component. The quantitative contribution of the peristaltic component can be calibrated by comparing calculations in accordance with the method of the first aspect with experimental data.

By doing so, the inventors demonstrated that canalicular peristalsis is a relevant contribution to bile flow; see Example 3.

As noted above, the structure of the mammalian liver at a mesoscopic level is defined by lobules. In a preferred embodiment, the method provides for predicting bile flow through (a) a single liver lobule; (b) a plurality of lobules, preferably adjacent lobules; or (c) an entire liver.

In a preferred embodiment (i) the parameters for steps (b) and (c) are obtained by measurements using (a) intravital microscopy (IVM) movie(s) or movie(s) obtained by non-invasive imaging methods, e.g. Raman microscopy or micro magnetic resonance imaging (micro-MRI), of one or more detectable bile tracer molecule(s) flowing through the bile canaliculi in vivo, preferably a fluorescent molecule such as 6-carboxyfluorescein-diacetate, said movie preferably being subjected to image analysis; (ii) the representation of step (d) is obtained from confocal microscopy; and/or (iii) the data for step (e) is obtained from electron microscopy.

This preferred embodiment specifies preferred means and methods for obtaining information, data and parameters to be fed into the computer-based method in accordance with the present invention.

Regarding electron microscopy (EM), data can be collected from other patients as well as also from post-operational or postmortem tissue material. Based on the measurement of those tissues, it is possible to perform calibration.

Regarding confocal microscopy, data can be collected from biopsies obtained from the patients. For estimation, data collected from other patient(s) or healthy human tissue can be used as well.

Regarding confocal microscopy in animal experiments, the best results are obtained when data is collected from the same animal. For calibration, confocal measurements from many different animal may be used. It was found that the data was highly reproducible. For human application, surgical tissue samples are preferred for data calibration.

As regards the tracer molecule in accordance with item (i), preference is given to tracer molecules which enter the blood circulation. In the alternative, tracers for bile flux can also be administered orally. A further example of useful tracers are fluorescent bile acid derivatives; see, for example, Holzinger et al., Hepatology 26, 1263 (1997).

To the extent intravital microscopy (IVM) or any other invasive method is to be used, it is understood that this is preferably not to be applied to humans, but instead animal models such as mice. Yet, there is evidence of uses of in vivo micro-MRI imaging in various human tissues, such as the bone with high spatial resolution (Li et al, Medical Physics, 35(12): 5584-5594 (2008)), and also the developing mouse vascular system (Berrios-Ottero, Magn Reson Med. 35(12): 5584-5594 (2009)).

The present preferred embodiment offers, as an alternative, also non-invasive methods which are accordingly applicable to all mammalian species including humans.

The suitability of non-invasive methods such as Raman microscopy or micro magnetic resonance imaging (micro-MRI) for determining integral bile flux is known in the art.

In particular, Raman microscopy allows to image fluorescent label-free tissue by monitoring the non-linear effect of scattered wavelength shift on molecular vibrational states. Usage of near far red lasers (750-900 nm) allows to detect Raman signal through 2-4 cm fat tissue (Matousek et al, J. Biophotonics, 6, 7-19 (2013), Ghita et al., J. Biophotonics, 11, 1-8 (2017); Meksiarun et al., Scientific Reports, 6:37068 (2016)) and was applied for non-invasive microscopy in medicine (see, e.g., Duraipandian et al, Analyst, 138, 4120-4128 (2013)). The far red (950-3000 nm) has the capacity to exploit the so called “second transparency window” of water (1200-1400 nm) with a wavelength that have ˜50 fold less scattering attenuation by Rayleigh mechanism. The necessary optical components, lasers and detectors are commercially available (e.g. from Ealing, ThorLabs, and Hamamatsu).

Resting-state whole brain functional magnetic resonance imaging (RFMRI) is an established tool for functional connectomics to characterize normal brain function and the various disorders with both high temporal and spatial resolution (see, e.g., Vu et al, Neurolmage 154, 23-32 (2017)).

While sufficient information about the canaliculi network is generally obtained from confocal microscopy, the fine structure inside the canaliculi is preferably analyzed using electron microscopy. As an alternative (or in addition) to electron microscopy, single molecule localization microscopy (SMLM) may be used.

In a preferred embodiment, the correction factor in accordance with step (e) of the method and to be obtained in accordance with item (iii) of the present preferred embodiment, is calculated once for a given species. In an alternative preferred embodiment, it is redetermined for each disease or disorder, or even for each individual.

In a particularly preferred embodiment, artifacts introduced by motion of the liver or part thereof during said intravital microscopy (IVM) or said non-invasive imaging methods are reduced or removed by embedding said liver or part thereof in vivo in a water-based gel and/or by correcting said IVM movies or movies obtained by non-invasive methods by image processing; see the section entitled “Correction and quantification of IVM movie shift” in Example 2.

In line with what has been said about the applicability of in vivo methods above, said embedding, in a preferred embodiment, is applied to non-human mammals.

In a preferred embodiment of the method, to the extent it includes calculating a peristaltic component of bile flow, the relative magnitude of said peristaltic component is determined by comparing bile flow calculated by said method to bile flow observed experimentally under conditions where osmosis and peristalsis coexist and/or conditions with perturbed acto-myosin contractility, for example upon administration of Fasudil.

To explain further, Fasudil is an agent which reduces or abolishes acto-myosin contractility. In other words, upon administration of Fasudil, bile flow will predominantly or exclusively be governed by osmosis.

In a further preferred embodiment of the method in accordance with the first aspect, and to the extent said method uses option (ii) for step (f), the parameters of said porous medium model, said parameters preferably being the anisotropic permeability tensor, are determined by fitting to experimental bile flow data.

In a second aspect, the present invention relates to use of (a) a three-dimensional representation of the bile canaliculi in a mammalian liver or a lobule thereof for computer-based prediction of bile flow; and/or (b) partitioning the axis connecting the central vein of a given lobule and a portal vein of said given lobule of a mammalian liver into a central zone, a middle zone and a portal zone, wherein bile transport in each zone is governed by zone-specific parameters.

As noted above, key aspects of the present invention are a precise three-dimensional representation of the canaliculi network and/or the recognition that within each lobule the definition of the above disclosed zones allows a more accurate modeling of bile transport.

Preferred embodiments of the three-dimensional representation and of defining said central zone, middle zone and portal zone, said preferred embodiments being disclosed in relation to the method of the first aspect, apply mutatis mutandis also to the remainder of the aspects of the present invention.

In a preferred embodiment of the method of the first aspect and the use of the second aspect, said ordinary differential equations of (c) are as follows:

$$\begin{matrix}\frac{{dC}_{{c}_{cv}}}{dt} = \frac{{V}_{cv} * {k}_{cleav} * {q}_{cv}}{( {q}_{cv} + {C}_{s} ) * {C}_{s}} + {k}_{{l}_{cv}} * {C}_{{b}_{cv}} - \frac{{k}_{pump} * q {1}_{cv}}{( q {1}_{cv} + {C}_{{c}_{cv} )} * {C}_{{c}_{cv}}} & ( Ia ) \\ \frac{{dC}_{{c}_{md}}}{dt} = \frac{{V}_{md} * {k}_{cleav} * {q}_{md}}{( {q}_{md} + {C}_{s} ) * {C}_{s}} + {k}_{{l}_{md}} * {C}_{{b}_{md}} - \frac{{k}_{pump} * q {1}_{md}}{( q {1}_{md} + {C}_{{c}_{md}} ) * {C}_{{c}_{md}}} & ( Ib ) \\ \frac{{dC}_{{c}_{pv}}}{dt} = \frac{{V}_{pv} * {k}_{cleav} * {q}_{pv}}{( {q}_{pv} + {C}_{s} ) * {C}_{s}} + {k}_{{l}_{pv}} * {C}_{{b}_{pv}} - \frac{{k}_{pump} * q {1}_{pv}}{( q {1}_{pv} + {C}_{{c}_{pv}} ) * {C}_{{c}_{pv}}} & ( Ic )\end{matrix}$$

for the cytoplasmic compartment, and:

$$\begin{matrix}\frac{{dC}_{{b}_{cv}}}{dt} = \frac{{k}_{pump} * q {1}_{cv}}{( q {1}_{cv} + {C}_{{c}_{cv}} ) * {C}_{{c}_{cv}}} - {k}_{{l}_{cv}} * {C}_{{b}_{cv}} - {k}_{{t}_{{cv}_{md}}} * {C}_{{b}_{cv}} & ( IIa ) \\ \frac{{dC}_{{b}_{md}}}{dt} = \frac{{k}_{pump} * q {1}_{md}}{( q {1}_{md} + {C}_{{c}_{md}} ) * {C}_{{c}_{md}}} - {k}_{{l}_{md}} * {C}_{{b}_{md}} + {k}_{{t}_{{cv}_{md}}} * {C}_{{b}_{cv}} - {k}_{{t}_{{md}_{pv}}} * {C}_{{b}_{md}} & ( IIb ) \\ \frac{{dC}_{{b}_{pv}}}{dt} = \frac{{k}_{pump} * q {1}_{pv}}{( q {1}_{pv} + {C}_{{c}_{pv}} ) * {C}_{{c}_{pv}}} - {k}_{{l}_{pv}} * {C}_{{b}_{pv}} + {k}_{{t}_{{md}_{pv}}} * {C}_{{b}_{md}} - {k}_{{t}_{{pv}_{out}}} * {C}_{{b}_{pv}} & ( IIc )\end{matrix}$$

for the bile canaliculi compartment,
wherein: Cₖᵢ are concentrations of bile or tracer molecule, wherein subscript k indicates the compartment in the lobule, k being either cytoplasmic (c) or bile canaliculi (b), and subscript i indicates the zone in accordance with step (a), i being central (cv), middle (md) or portal (pv); Vᵢ are volumes of respective zones; qᵢ are metabolic activity rates within respective zones; q1ᵢ are bile secretion rates within respective zones; kₜᵢ are transport rates between respective zones; kₗᵢ is the rate of leakage of the apical membrane in the respective zones (k_{lcv}, k_{lmd}, kₗₚᵥ); k_{cleav} is the conversion rate of a non-detectable form of said tracer molecule into a detectable form; and kₚᵤₘₚ is the transport rate of bile across the apical plasma membrane of hepatocytes, i.e. the specialized surface of hepatocytes that forms the bile canaliculi;
and wherein

$$\begin{matrix}{v}_{cv} = \frac{{k}_{{t}_{{cv}_{md}}} * {V}_{cv}}{{A}_{cv}} * L & ( IIIa ) \\ {v}_{md} = \frac{{k}_{{t}_{{md}_{pv}}} * {V}_{md}}{{A}_{md}} * L & ( IIIb ) \\ {v}_{pv} = \frac{{k}_{{t}_{{pv}_{out}}} * {V}_{pv}}{{A}_{pv}} * L & ( IIIc )\end{matrix}$$

wherein: vᵢ are bile fluid velocities at the interface or transition between zones; kₜᵢ are transport rates between respective zones; Vᵢ are volumes of respective zones; Aᵢ are boundary surfaces of respective zones; and L is the distance between said central vein and said portal vein.

A tracer, preferably a fluorescent tracer is used to experimentally determine bile flux. A preferred fluorescent tracer is CF, and its non-fluorescent form is CFDA. Flux of said tracer is the same as the flux of bile. In order to account for the properties of the tracer, k_{cleav} is used. In case of CFDA, k_{cleav} is the cleavage rate of CFDA by cytoplasmic esterases. Cleavage yields CF. The term “leakage” refers to the passive backflow of tracer molecules from the canaliculi lumen into the hepatocyte.

The apical side of hepatocytes faces the lumen of the bile canaliculi.

In a further preferred embodiment of the method of the first aspect and the use of the second aspect, to the extent step (f-i) as defined in the first aspect is performed, said spatial profile of (g) is as follows:

$$v ( x ) = \frac{RTc}{2 μ} \sqrt{a ( x ) κ μ} \frac{sinh ( \sqrt{\frac{16 κ μ {L}^{2}}{{a ( x )}^{3}}} \frac{x}{L} )}{cosh ( \sqrt{\frac{16 κ μ {L}^{2}}{{a ( x )}^{3}}} )} \qquad ( IV )$$

wherein: v(x) is the bile fluid velocity at a given coordinate x; x is the coordinate along the axis defined in (a), x being 0 at the central vein and L at the bile duct; a(x) is hydraulic radius according to step (e) radius at given coordinate x; R is the universal gas constant; T is temperature; μ is the fluid viscosity of bile; κ is the water permeability of the apical membrane of hepatocytes; c is a scale factor; and L is as defined above.

In a particularly preferred embodiment, parameters c and K in equation (IV) are determined by fitting to velocity at the boundaries of zones as defined by equations (IIIa) to (IIIc) above.

The parameter κ (water permeability) depends on details of lipid/protein composition of apical membranes of hepatocytes, their area and curvature. This parameter is found by fit of mesoscopic flux to the experimental data. The scale factor c accounts for the difference in geometry of lobule where experimental measurements of flux were performed and the geometry of an idealized hexagonal lobule.

This preferred embodiment provides for a separate calculation of the flux of bile or tracer, respectively, for the cytoplasmic compartment, i.e. within hepatocytes, and the bile canaliculi compartment. In particular, the derivatives with respect to time of the concentrations in accordance with equations (Ia) to (Ic) and (IIa) to (IIc) define fluxes.

The volumes of the respective zones are preferably calculated on the basis of a geometrical model obtained from biopsy samples of healthy, untreated and/or perturbed liver tissue, e.g. from the three-dimensional representation of (d).

Further parameters of the equations, to the extent necessary, may be determined by fitting the solutions of said equations to experimental data obtained by intravital and/or Raman microscopy.

Equation (IV) describes the profile of bile velocity along the portal vein-central vein axis. It is a solution to the Navier-Stokes equation for laminar Newtonian liquid in the representation of the bile canaliculi of (d) with for the fluxes of equations (Ia) to (Ic) and (IIa) to (IIc) and the fluid velocities at the zone boundaries in accordance with equations (IIIa) to (IIIc).

In a third aspect, the present invention provides a computer program comprising instructions to cause a computer to execute the steps of the method of the first aspect.

In a fourth aspect, the present invention provides a computer-readable medium (a) comprising instructions which, when executed on a computer, cause said computer to execute the steps of the method of the first aspect; and/or (b) having stored thereon the computer program in accordance with the third aspect.

In a fifth aspect, the present invention provides a computer comprising means for carrying out the method of the first aspect, such means preferably being the computer program in accordance with the third aspect and/or the computer-readable medium in accordance with the fourth aspect.

In a sixth aspect, the present invention provides the use of the method of the first aspect, the computer program of the third aspect, the medium of the fourth aspect or the computer of the fifth aspect (a) for predicting bile flow upon administration of an agent, lead compound or drug; (b) for predicting drug-induced liver injury by an agent, lead compound or drug; (c) in silico safety assessment of an agent, lead compound or drug; (d) in diagnosis, in particular of cholestatic subtypes; (e) in personalized medicine; or (f) for determining the quantitative contribution of peristalsis to bile flow.

It is known in the art that a number of pharmaceutically active agents (for reviews see, for example Chalasani et al., American Journal of Gastroenterology, 1-17 (2014); Padda et al., Hepatology, 53, 1377-1387 (2011); and Haque et al., Gut and Liver, 10, 27-36 (2016)) including anti-inflammatory agents modify bile flow and may, in particular at high dosages, cause drug-induced liver injury (DILI). In view of the high accuracy of the means and methods in accordance with the present invention, such effects of an agent, lead compound or drug can be predicted.

Related thereto, the present invention can be used for an in silico safety assessment of an agent lead compound or drug. For example, when a choice is to be made prior to drug development among a number of lead compounds for the treatment of a specific medical indication, in silico drug profiling (or lead profiling) in accordance with the present invention may be helpful for decision making.

As regards item (d) of the sixth aspect, we refer to the explanations given above, namely that the model can be fed with the data, information and parameters specific for a given individual. Accordingly, the present invention can be used in the sense of stratifying the population and personalizing diagnosis and treatment.

In relation to the use in accordance with the sixth aspect, parameters of particular relevance and informative character are the following: (i) the distribution of the bile canaliculi diameter along the axis from the central vein to the portal vein, (ii) the flux of bile across zone boundaries, zones being defined in accordance with item (a) of the first aspect, and (iii) features of the three-dimensional network of bile canaliculi such as the density of the canaliculi.

In a seventh aspect, the present invention provides a method of diagnosing a predisposition for developing cholestasis, liver steatosis, liver fibrosis and/or liver cirrhosis, said method comprising the method of the first aspect, said developing preferably being in response to a disease state or administration of an agent, lead compound or drug.

In a preferred embodiment of the method of the seventh aspect, said method comprises providing a three-dimensional representation of the bile canaliculi of the patient for whom said predisposition is to be determined. This preferred embodiment illustrates further the notion of personalized medicine disclosed above.

In a further preferred embodiment of the method of the seventh aspect or the use of the sixth aspect, wherein said agent or drug is selected from agents which interfere with the actin cytoskeleton or acto-myosin contractility such as Fasudil; antimicrobials such as isoniazid, rifampin, pyrazinamide, amoxicillin-clavulanate, sulfonamides, nitrofurantoin, minocycline, and ketoconazole; antiretrovirals such as NRTIs, nNRTIs, and protease inhibitors; antiepilectics such as phenytoin, carbamezapine, and valproic acid; analgesics such as NSAIDs including acetoamiphen; lipid lowering agents including statins; immunologics including TNF antagonists; herbal and dietary supplements such as ephedra, green tea extract, and muscle enhancers.

In the following, a more detailed account is given of preferred implementations of the elements of the model and of the steps of the method in accordance with the first aspect of the present invention.

General Strategy of a 3D Multi-Scale Model of Biliary Fluid Dynamics

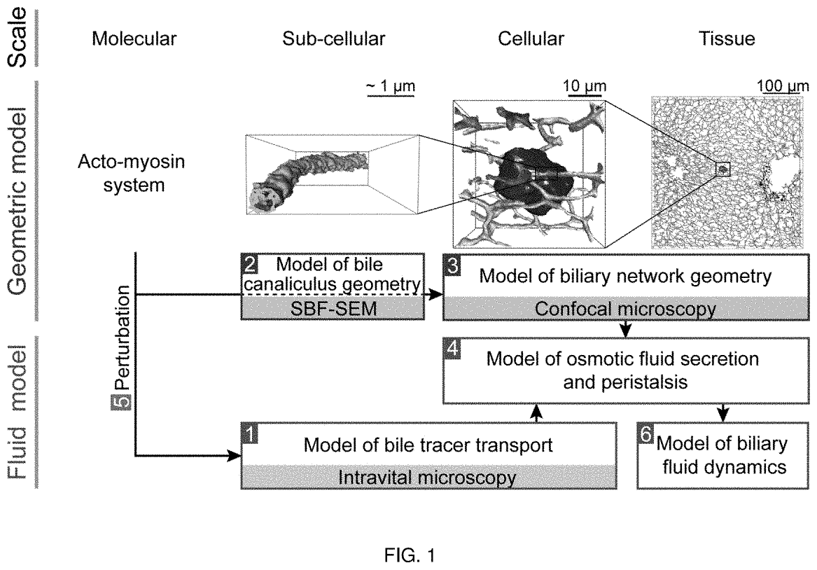

BC form a contiguous tubular network throughout the liver lobule that transports bile. Bile transport is generally thought to depend on the osmotic effect of bile secretion (Ballatori and Truong, 1989; Boyer and Bloomer, 1974; Boyer and Klatskin, 1970). However, a peristaltic mechanism has also been evoked (Oshio and Phillips, 1981; Watanabe et al., 1991). To simulate biliary fluid dynamics, we successively integrated the structural and functional properties of the BC network from each level of organization, sub-cellular (i.e. the apical membrane of hepatocytes), cellular (the hepatocytes) and tissue (the BC network within the lobule), into a 3D multi-scale model following the strategy schematically illustrated in FIG. 1. First, we determined sub-cellular parameters of bile transport by IVM and mathematical modelling of a fluorescent bile tracer (step 1). Next, we complemented these functional measurements with quantitative models of BC network geometry obtained from a combination of electron and confocal microscopy (step 2 and 3). From these measurements, we determined the parameters of bile flow through the BC network (step 4). Using pharmacological perturbation of acto-myosin contractility, we determined the contribution of osmosis and peristalsis to biliary flow (step 5). Finally, we combined experimental and modelling results in a predictive tissue-scale model of biliary fluid dynamics (step 6).

Intravital Imaging of the Bile Tracer CFDA Reveals Heterogeneities of Hepatic Transport within the Liver Lobule

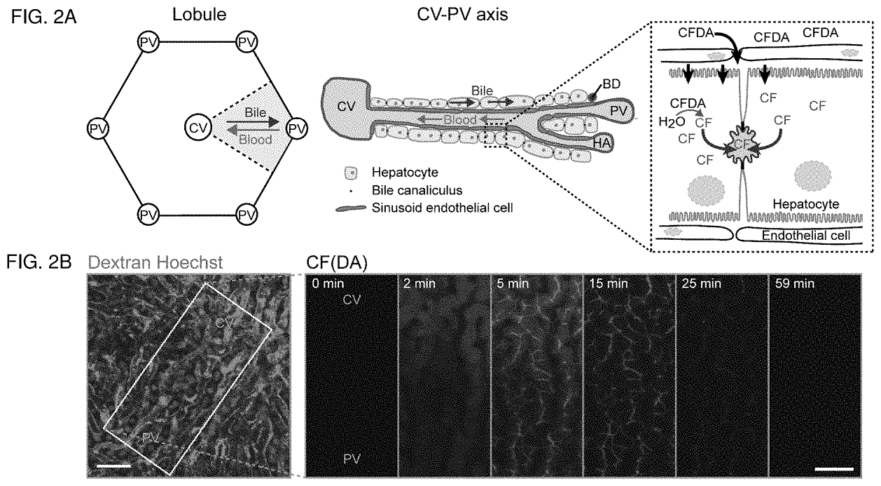

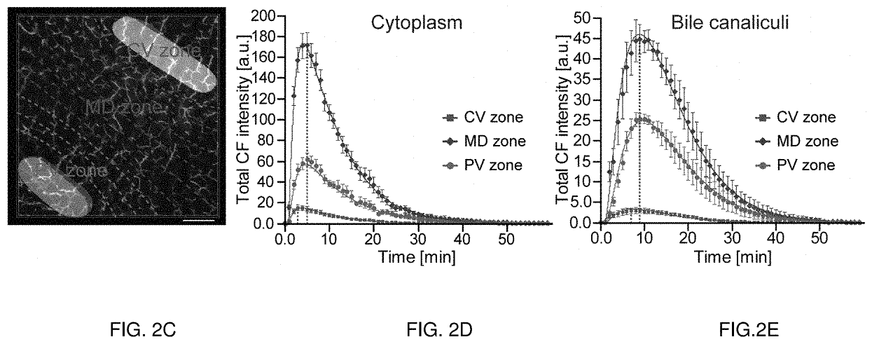

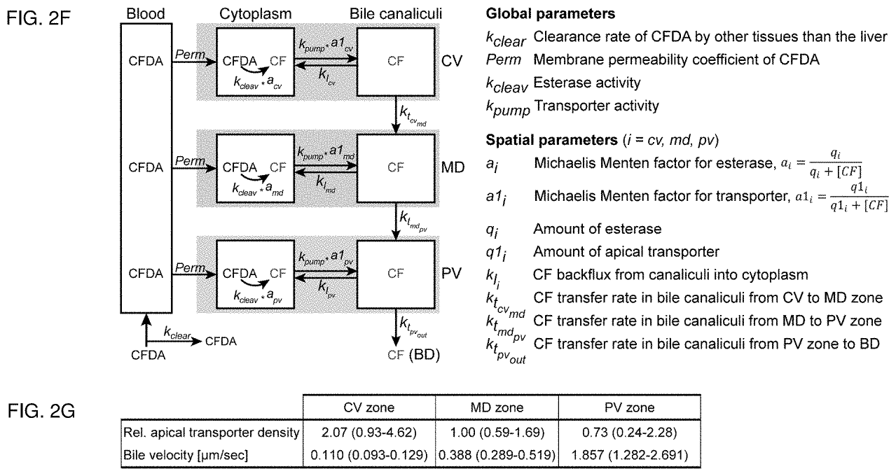

The osmotic effect of hepatocyte apical secretion represents the main determinant of bile flow. It depends on the secretion of bile salts (bile salt dependent bile flow, BSDF) (Boyer and Bloomer, 1974) and other organic molecules such as glutathione (bile salt independent bile flow, BSIF) (Ballatori and Truong, 1992; Boyer and Klatskin, 1970). Due to the functional zonation of the liver lobule, (Baier et al., 2006; Berkowitz et al., 1995; Groothuis et al., 1982), an insight into the osmotic effects of bile secretion would require a spatially differentiated analysis of the collective apical transport activities of hepatocytes, which is currently impossible. To derive an estimate of apical bile secretion and bile velocity in the BC network we performed IVM of the well-established fluorescent tracer 6-carboxyfluorecein-diacetate (CFDA) (Babbey et al., 2012; Breeuwer et al., 1995; Liu et al., 2007; Porat-Shliom et al., 2016). CFDA secretion depends on the apical multidrug resistance-associated protein 2 (MRP2) (Zamek-Gliszczynski et al., 2003) which primarily transports organic anions (BSIF) (Paulusma et al., 1999; Wielandt et al., 1999) and only to a minor extent glucuronidated bile acids (BSDF) (Akita et al., 2001). CFDA is a membrane permeant molecule that becomes fluorescently excitable and membrane impermeable upon hydrolysis into 6-carboxyfluorescein (CF) by intracellular esterases (Breeuwer et al., 1995). Following intravenous (i.v.) injection, CFDA enters the liver lobule through the blood via the PV and drains into the CV. Along this portocentral axis, CFDA is taken up by hepatocytes, cleaved into its fluorescent derivative CF in the hepatocyte cytoplasm, actively secreted into the BC by MRP2, and cleared from the system via the common bile duct (FIG. 2A) (Babbey et al., 2012; Breeuwer et al., 1995; Zamek-Gliszczynski et al., 2003).

To take into account heterogeneities of bile transport within the liver lobule, we imaged CFDA transport in a spatially resolved manner along the entire porto-central axis in adult mice (step 1 in FIG. 1). A typical problem in such experiments are the artefacts introduced by motion. To minimize cardiac and respiratory motion artefacts, we embedded the organ in a water-based gel in a custom designed microscope stage-insert (see Method details in Example 1 and (Porat-Shliom et al., 2016)). Particular care was used to avoid artefacts caused by surgery and photo-toxicity. This is especially important when IVM is conducted under perturbations (see below). Our set-up sustained physiological levels of blood flow and did not present evidence of apparent tissue damage in the exposed liver lobe, proving high stability without compromising tissue integrity. Remaining sample drift was corrected by development and implementation of new image processing algorithms into the MotionTracking software ((Morales-Navarrete et al., 2015) http://motiontracking.mpi-cbg.de, see Example 2, section ‘Correction and quantification of IVM movie shift’).

To identify the CV-PV axis of a liver lobule, the direction of blood flow and sinusoid orientation was visualized by i.v. injection of fluorescent-conjugated dextran (FIG. 2B). Following image acquisition start, CFDA was administered i.v. and detectable in the hepatocyte cytoplasm within seconds (FIG. 2B). Subsequently, CF became visible in the BC network within 3-5 min and cleared from the system within ˜40 min. Comparison of intracellular fluorescence intensities along the CV-PV axis 2 min after CFDA injection revealed that peri-central hepatocytes accumulated the tracer faster than peri-portal hepatocytes (FIG. 2B). Such heterogeneities and the observed transport dynamics are in agreement with previous reports of CF(DA) transport in the liver (Babbey et al., 2012; Lin et al., 2016).

Bile Velocity Establishes a Gradient within the Porto-Central Axis

To estimate bile transport rates, we quantified the tracer intensities from IVM movies and described its kinetics in a mathematical model (step 1 in FIG. 1). To determine the spatial heterogeneities of CF(DA) transport, we computationally divided the porto-central axis into three zones, the central zone (CV) corresponding to approximately one cell layer, the middle (MD) with ˜six and portal (PV) zone with ˜three cell layers (FIG. 2C), similar to the metabolic zonation (Benhamouche et al., 2006; Jungermann, 1988; Jungermann and Kietzmann, 1996; Morales-Navarrete et al., 2015). Within each zone, we quantified the CF intensity in the hepatocyte cytoplasm and BC. Both compartments were segmented automatically from IVM movies using an ad hoc developed algorithm implemented in the MotionTracking software (see Example 2, section ‘Quantification of CF intensities from intravital movies’). As expected from the zonation of CF secretion and the direction of bile flow, CF was secreted and cleared faster in the BC network in the CV zone than the MD and PV zones (FIG. 2D, E).

Next, we described the tracer kinetics in a mathematical model formulated as a set of ordinary differential equations (ODE) to estimate bile transport rates (FIG. 2F, see Example 2, section ‘Mathematical 3-compartment model of CF(DA) transport in the liver’). The model distinguishes tracer transport for the CV, MD and PV zones and considers the different sizes and contact interfaces of the kite-shaped CV-PV area of the lobule (see FIG. 2A). The model considers that, within each zone, CF is transported sequentially from the blood into the hepatocyte cytoplasm and, from here, actively secreted into the BC network. In addition, passive back flux from the BC into the cytoplasm is included. While CFDA rapidly equalizes between blood and hepatocytes cytoplasm by passive diffusion (Breeuwer et al., 1995), the (fluorescent) cleaved form CF is membrane impermeant and requires active transport. Therefore, the model assumes that both esterase activity and apical secretion follow Michaelis-Menten kinetics. To consider potential zonation of intracellular esterases and CF apical transporters, the model accounts for spatially heterogeneous levels of both parameters. For the BC compartment, the model considers that the networks of the three lobule zones are connected and that CF propagates from the CV to the MD and PV zone network via the bile flow. Finally, CF exits the BC network from the PV zone via the bile duct. Fitting of the model to the experimental measurements of CF intensities (FIG. 2D, E, solid lines) showed that hepatic CF transport differs significantly within the liver lobule (FIG. 2G). First, consistent with (Lin et al., 2016), the density of apical CF transporters, and thus the apical secretion rate, was ˜2.8-fold higher in the CV zone than in the PV zone. Second, bile velocity within the BC network increased progressively from the CV to the PV zone. Across the long distance of the MD zone (about 6 cell layers in diameter), bile velocity raised gradually about 3.5-fold. In the distal peri-portal network, however, within a distance of only 3 cells layers, bile velocity increased dramatically 4.8-fold from 0.388 μm/sec to 1.857 μm/sec. These results demonstrate for the first time the quantitative differences of biliary fluid dynamics within the BC network of the liver lobule.

A Mechanistic Model of Osmotic Fluid Secretion and Bile Flow

The osmotic effect on bile flow depends on the apical secretion of bile by hepatocytes and the geometry of the BC network. To explain the gradient of bile velocity along the CVPV axis, we estimated the spatial distribution of osmotic water influx into the BC (step 4 in FIG. 1) from measurements of BC geometry (step 2 and 3 in FIG. 1) and hepatocytes apical CF-secretion.

To determine the geometrical properties of the BC network we used our previously reported digital 3D model (step 3 in FIG. 1, (Morales-Navarrete et al., 2015)). An important feature of this model is that it provides an accurate and spatially resolved estimate of the BC network parameters which are crucial for the spatial fluid dynamics model. We reconstructed the BC network from confocal images of immuno-stained thick liver tissue sections using the MotionTracking software (Morales-Navarrete et al., 2015). Sections were stained with the hepatocyte apical marker CD13 and an entire CV-PV axis was imaged as high-resolution z-stacks (approx. 400×230×80 μm in xyz dimension) (FIG. 3A). Image stacks were used for 3D network reconstructions (FIG. 3B) and the BC radius was calculated for 20 zones along the CV-PV axis. The results demonstrate that the BC radius is variable, being smallest in the middle of the CV-PV axis (1.03 μm±0.05) and increasing up to 1.3-fold towards the CV and PV areas (FIG. 3C, left y-axis).

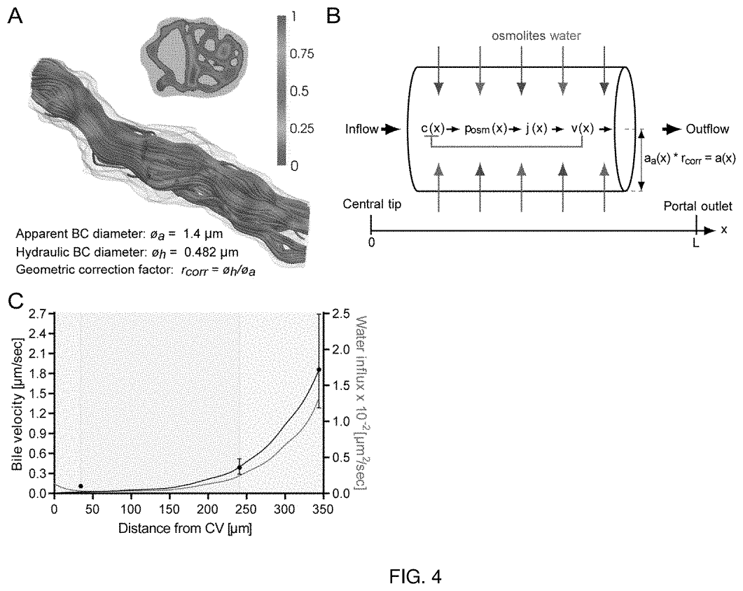

Importantly, BC lumens are convoluted and densely packed with microvilli. Clearly, such a micro-architecture is not resolvable by confocal microscopy, but affects coarse-grained fluid dynamic properties of bile flow such as friction. To account for this, we performed 3D electron microscopy (EM) analysis of liver tissue to reconstruct a BC and calculate its hydraulic diameter, a measure for the cross-sectional flow in non-circular tubes (step 2 in FIG. 1). We performed serial block-face scanning EM (SBF-SEM) (Deerinck et al., 2010) which allows for the automated image acquisition of relatively large volumes of tissue at nanometer resolution (12.4 nm pixel size, 40 nm steps in z dimension) (FIG. 3D). Plane-wise manual segmentation of EM image stacks followed by 3D reconstruction and surface mesh generation using the Imaris software (BitPlane, Inc.) visualized the dense packing of microvilli (FIG. 3E). In the light of these structural features, BC cannot be considered as smooth circular tubes in the model. This is accounted for by the first correction factor as defined in the method of the first aspect. To determine the relation between bile velocity and pressure within the reconstructed BC, the Navier-Stokes equations were numerically solved in the 3D reconstructed BC geometries. Simulations were carried out using the finite-volume method for physiologically relevant bile pressure conditions (10⁻³-10⁵ Pa). Bile is an incompressible Newtonian fluid with a viscosity similar to water (Luo et al., 2007). Given its low Reynolds number (Re ˜10⁻⁶), bile flow can be considered to be laminar (see also Example 2, section ‘Computational fluid dynamics simulation of bile flow in a 3D EM-reconstructed BC’). From the simulations of bile flow in the reconstructed BC (FIG. 4A) we found a hydraulic diameter of 0.482 μm which was significantly smaller than its average apparent diameter of 1.4 μm (as measured from EM images). Applying this ratio as a correction factor (r_{corr}=0.344) to our confocal microscopy-based reconstruction we could derive realistic estimates of bile flow from the digital 3D geometric model.

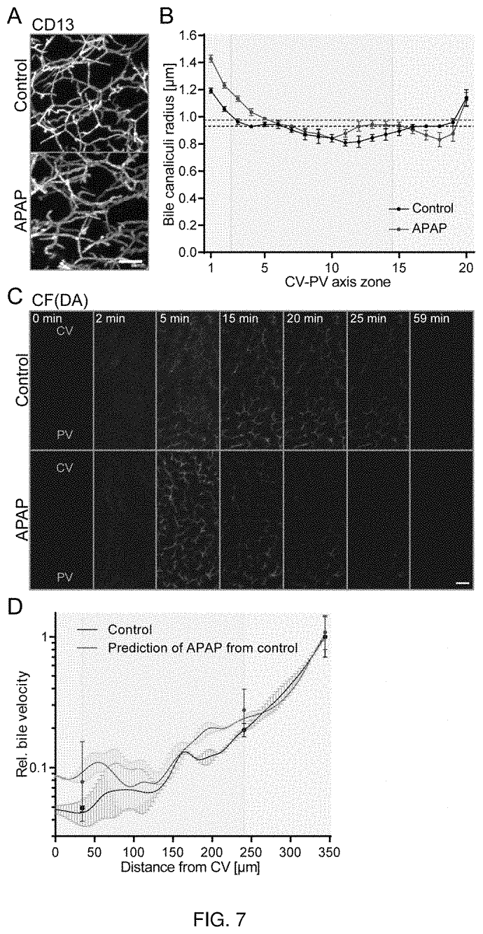

Based on our geometric model and IVM measurements of apical transport we developed a mechanistic model of osmotic bile secretion and bile flow (step 4 in FIG. 1). The model considers the measured heterogeneous BC radius profile along the porto-central axis (FIG. 3C), rescaled to the equivalent hydraulic radius, an average BC path length of 344 μm between the CV and PV area (measured from the geometric model), the different sizes and interfaces of the zones within the kite-shaped CV-PV axis as well as previously reported fluid mechanic properties of bile (Luo et al., 2007; Mathias, 1985). Mechanistically, the model assumes that the secretion of osmolites into the BC network generates an osmotic pressure that drives water influx and, consequently, bile flow (FIG. 4B). The osmotic pressure is counteracted by the local fluid pressure and negatively regulated by the loss of osmolites from fluid outflow. We solved the model analytically for steady state flow conditions assuming zero flow at the origin of the network in the CV zone and zero pressure at the open outlet in the PV zone (see Example 2, section ‘Mechanistic model of osmotic fluid secretion and bile flow’). The spatial profile of the bile flow velocity v(x) was obtained as a function of the measured BC radius a(x) and the CV-PV axis distance L, and two parameters p₁ and p₂, representing combinations of material properties of the liver. The parameters p₁ and p₂ were determined by fitting the analytical function v(x) to the experimental data points. The resulting bile velocity and BC network water influx profiles revealed an exponential-like increase from the CV to the PV area (FIG. 4C). Overall, the model result reproduced well our IVM measurements in the MD and PV zones, although underestimated the peri-central bile velocity. This suggested that bile flow could be driven by an additional mechanism, e.g. BC peristalsis (Oshio and Phillips, 1981; Watanabe et al., 1991). We therefore set out to test the function of peristalsis for bile flow and, finally, validate the predictive capacity of the model (see Example 5, entitled ‘Sub-toxic doses of acetaminophen affect biliary fluid dynamics’ and FIG. 7D).

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show:

FIG. 1: Strategy of a multi-scale model of biliary fluid dynamics

Strategy for the development of a multi-scale model of biliary fluid dynamics by integration of geometric and fluid dynamic models from different scales. Bile canaliculi (BC) network organization: The acto-myosin system (molecular scale) is a central component of the sub-apical cortex of hepatocytes that form BC from their apical membrane (sub-cellular scale). These build continuous belts in between neighboring hepatocytes (cellular scale) and a ramified tubular network throughout the liver (tissue scale). Modelling strategy: Biliary transport properties were estimated from intravital microscopy of a bile tracer in a (sub-)cellular model of bile transport (step 1). Next, geometric BC properties were quantified from 3D models of the single bile canaliculus (step 2) and BC network (step 3) using serial block face-scanning electron (SBF-SEM) and confocal microscopy. Based on these experimental measurements, parameters of bile flow were determined in a model of osmotic fluid secretion and biliary peristalsis (step 4) using molecular perturbation of the acto-myosin machinery (step 5). The model result was applied to simulate biliary fluid dynamics at the tissue level (step 6).

FIG. 2: Quantification of bile transport by intravital imaging of CFDA

A) Schematic illustration of the liver lobule geometry and hepatic CF(DA) transport. In the lobule, blood flows from the portal vein (PV) to the central vein (CV), whereas bile flows counter current and drains into the bile duct (BD). CFDA is taken up by hepatocytes from the blood, cleaved into its fluorescent derivative CF in the cytoplasm, secreted into BC and transported through the BC network via the bile flow; HA, hepatic artery B) Representative images from an IVM movie showing the transport of CF(DA) within the CV-PV axis at indicated time points after acquisition start. CFDA was administered at 1 min. The approximate localization of the CV and PV was identified from the vascular flow patterns using fluorescent-conjugated dextran. Shown are maximum projections of 19 μm z-stacks. C) Schematic illustration of the spatial analysis approach of CF transport from IVM movies. The localization of the CV and PV was estimated from the vascular flow patterns, the CV-PV axis was computationally divided into 10 equidistant areas (dashed lines) and the CF intensities were quantified in the CV, MD and PV zones. D, E) Quantification of the total CF intensity in the hepatocyte cytoplasm (D) and bile canaliculi (E) from IVM movies as shown in (B), determined for the CV (squares), MD (diamonds) and PV (circles) zones. Dots represent experimental measurements (n=4 mice, mean±68% CI), solid lines show the fit of the 3-compartment model of CF(DA) transport shown in (F). Dashed lines indicate time points of maximum CF intensity. a.u., arbitrary units of intensity. F) Schematic representation of the compartment model of CF(DA) transport in the liver. For model description, see text. Rate constants are described on the right. Model results are displayed as solid lines in (D) and (E). G) Relative apical transporter density and bile velocity estimated from IVM of CF transport using the model shown in (F). Transporter density was determined based on the kite-shaped geometry of the CV-PV axis and is expressed relative to the MD zone. n=4, mean±95% CI. Scale bars: 50 μm (B,C).

FIG. 3: A geometric model of the BC network reveals the structural heterogeneity within the liver lobule

A) Representative IF images of fixed mouse liver tissue sections stained for the apical marker CD13. Shown is a maximum projection of a 78 μm z-stack covering an entire CV-PV axis (CV to the left, PV to the right). B) 3D reconstruction of the BC network shown in (A). The CV is shown on the left, the PV on the right. C) Quantification of bile canaliculi radius (left y-axis) and tissue porosity (right y-axis) from reconstructions as representatively shown in (B). Quantification was performed for 20 equidistant zones along the CV-PV axis. Zone 1 and 20 are adjacent to the CV and PV, respectively, and displayed on the x-axis. The three segments in the background indicate the localization of the CV, MD and PV zones. n=3 mice, mean±SEM. D) Representative serial block face-scanning electron microscopy image stack of fixed mouse liver tissue. Shown is one XY plane of a 8 μm z-stack and YZ and XZ stack projections at the indicated lines (grey). E) 3D reconstruction of the bile canaliculus shown in (D). Scale bars: 100 μm (A, B), 2 μm (D).

FIG. 4: A mechanistic model of osmotic fluid secretion

A) Simulation of bile flow in the 3D geometry of the reconstructed BC shown in FIG. 3E. Bile velocity is expressed relative to the maximum and shown in the cross-sectional and longitudinal-plane of the BC. Values are color-coded as indicated by legend. The BC has a measured apparent diameter øa of 1.4 μm and a computed hydraulic diameter øh of 0.482 μm. The ratio of øh and øa defines the geometric correction factor r_{corr} that is used in the model of osmotic fluid secretion in (B). B) Mechanism of water influx into the BC network for the model of osmotic fluid secretion. Schematic drawing of a bile canaliculus with apparent radius aₐ(x), located between the tip of the BC network in the CV area (left) and the outlet in the PV area (right). The radius a(x)=aₐ(x)*r_{corr} is corrected for the hydraulic diameter of a BC as determined in (A) using the correction factor r_{corr}. In the BC network, bile is secreted and flowing along a series of branches with an average cumulative length L. The osmolite concentration profile c(x) generates the osmotic pressure surplus pₒₛₘ that drives water influx j(x) into the lumen. Osmotic pressure is counteracted by the local fluid pressure p(x) (not depicted) which is monotonously decreasing from a self-organising pressure maximum at the closed tip to zero at the open outlet. As bile flows out with cross-section averaged velocity (x), the osmolite concentration gets diluted, representing a negative feedback loop between fluid flow and osmotic driving (grey arrow). C) Prediction of the bile velocity (black, left y-axis) and water influx density (grey, right y-axis, unit: volume per time and per BC length) into the BC network along the CV-PV axis from the model of osmotic fluid secretion shown in (B). Solid lines show the model prediction, dots (black) represent experimental measurements of bile velocity (n=4, mean±95% CI) shown in FIG. 2G. The three segments in the background indicate the localization of the CV, MD and PV zones, respectively.

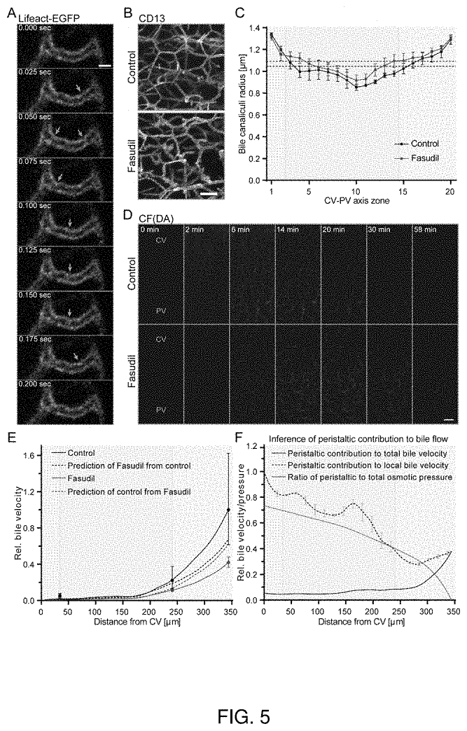

FIG. 5: Bile canaliculi contractility is a determinant of bile flow

A) Representative images from an IVM movie of a Lifeact-EGFP mouse liver showing the sub-apical F-actin belt of a BC. Images were acquired at the indicated time points. Arrows indicate BC constriction events. B) Representative IF images of fixed liver tissue sections from control and Fasudil-treated mice, stained for the apical marker CD13. Shown are maximum projections of 75 μm z-stacks acquired in the PV area. C) Quantification of the BC radius in control (black) and Fasudil-treated (grey) mice for 20 equidistant zones along the CV-PV axis. Zone 1 and 20 are adjacent to the CV and PV, respectively. Dashed lines represent the average network radius in control (black) and Fasudil-treated (grey) mice. n=3 mice, mean±SEM. Radius of control vs. Fasudil-treated mice, p>0.05 (CV zone), p<0.0001 (MD zone), p<0.001 (PV zone). D) Representative images from IVM movies showing the transport of CF(DA) in the mouse liver of control or Fasudil-treated mice at the indicated time points after acquisition start. CFDA was injected at 1 min. Shown are maximum projections of 17 μm z-stacks. The approximate localization of the CV and PV are indicated. Movies were acquired with identical imaging settings and are displayed with the same intensity threshold values. E) Test of the mechanistic model of osmotic fluid secretion. The model of osmotic fluid secretion was fit to experimental measurements of control (black solid line) and Fasudil-treated mice (grey solid line). From these model fits, bile velocity of Fasudil-treated mice was predicted from the control condition (black, dashed line) and bile velocity of control mice was predicted from Fasudil-treated mice (grey, dashed line) (see text for detailed description). The velocity axis is relative to the value of the control condition in the PV area. Dots represent experimental measurements (control, black; Fasudil, grey). n=4 mice (control), n=5 mice (Fasudil), mean±95% CI. Prediction of control from Fasudil at PV outlet vs. experimental measurement in control mice at PV outlet, p=0.09; Prediction of Fasudil from control at PV outlet vs. experimental measurement of Fasudil-treated mice at PV outlet, p<0.0001. F) Inference of the peristaltic contribution to bile flow. Shown is the peristaltic contribution to the total (black, solid line) and local (black, dashed line) bile velocity profile and to the total biliary pressure (grey, solid line). The contribution to total bile velocity was inferred from the difference between the model prediction (E, grey, dashed line) and an empirical fit of the experimental control measurements (not shown in E). The local contribution of peristalsis to bile velocity was inferred from the ratio of the peristaltic contribution to the total bile velocity. Pressure is expressed relative to the max. pressure obtained at the central tip. Error bars were propagated from the radius profile of control mice in (C). Peristaltic contribution to local bile velocity in CV vs. PV zone, p<0.0001. The three segments in the background in panel C, E and F indicate the localization of the CV, MD and PV zone, respectively. Scale bars: 2 μm (A), 10 μm (B), 25 μm (D).

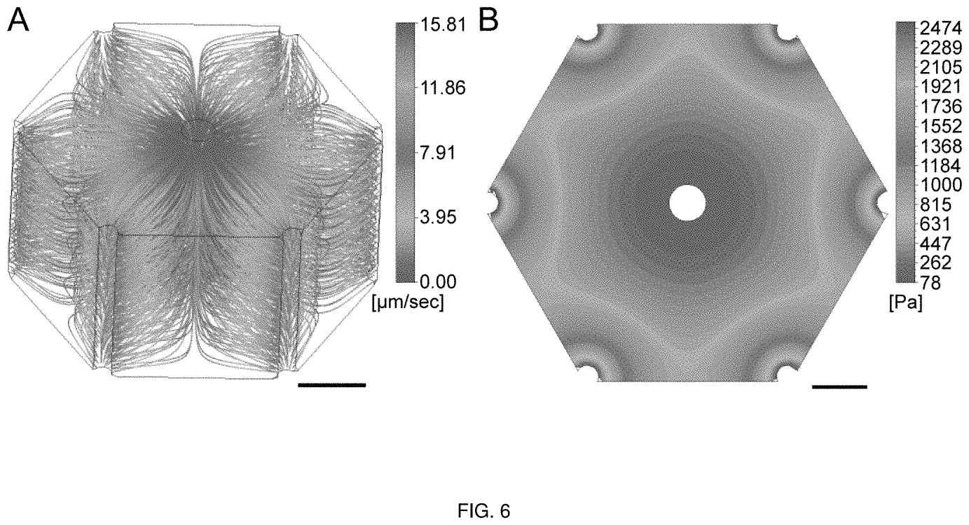

FIG. 6: A 3D porous medium model of biliary fluid dynamics

Simulation results of bile velocity (A) and bile pressure (B) from the anisotropic porous medium model. Velocity streamlines (A) and pressure profile (B) are shown in the hexagonal liver lobule geometry (CV is located in the center, PVs in the periphery). Values are color-coded as indicated by legend. Scale bar: 100 μm (A, B).

FIG. 7: Acetaminophen alters BC geometry and bile transport at a subtoxic dose

A) Representative IF images of fixed liver tissue sections in control or acetaminophen (APAP) treated mice, stained for the apical marker CD13. Shown are maximum projections of 78 μm z-stacks taken in the CV area. B) Quantification of BC radius (y-axis) of control and APAP-treated mice in 20 equidistant zones along the CV-PV axis (x-axis). Zone 1 and 20 are adjacent to the CV and PV, respectively. Average network radii are displayed as dashed lines (control, black; Fasudil, grey). Note: BC radius is smaller than in FIGS. 3C and 5C because tissue was immersion fixed (see Example 1). n=3 mice, mean±SEM. Radius of control vs. APAP-treated mice, p<0.01 (CV zone), p<0.0001 (MD zone), p>0.05 (PV zone). C) Representative images from an IVM movie of CFDA transport in control and APAP-treated mice at indicated time points after acquisition start. CFDA was injected at 1 min. Shown are maximum projections of 17 μm z-stacks. The approximate localization of the CV and PV are indicated. Movies were acquired with identical imaging settings and are displayed with the same intensity threshold values. D) Prediction of bile velocity in APAP-treated animals from the combined model of osmotic fluid secretion and peristalsis. The model was fit to experimental measurements of the control condition (black, solid line) and the resulting velocity profile was used to predict velocity upon APAP treatment (grey, solid line). Velocity is expressed relative to the value of control condition at the portal end and displayed in log-scale. n=4 mice (control), n=5 mice (APAP), mean±95% CI. Error bars of model results were propagated from the radius profiles shown in (B) and extrapolated across the CV-PV axis. Experimental measurements from APAP mice vs. model prediction of APAP from control, p=0.32 (CV zone), p=0.22 (MD zone), p=0.41 (PV zone). The three segments in the background in panel B and D indicate the localization of the CV, MD and PV zones, respectively. Scale bars: 10 μm (A), 25 μm (C).

The examples illustrate the invention.

### Example 1

Materials and Experimental Methods

Mouse Work

Animal experiments performed at the National Institutes of Health (Bethesda, Md., USA) were approved by the National Institute of Dental and Craniofacial Research (NIDCR, National Institutes of Health, Bethesda, Md., USA) Animal Care and Use Committee. Animal experiments performed a the Max Planck Institute of Molecular Cell Biology and Genetics (MPI-CBG, Dresden, Germany) were conducted in accordance with German animal welfare legislation and in strict pathogen-free conditions in the animal facility of the MPI-CBG, Dresden, Germany. Protocols were approved by the Institutional Animal Welfare Officer (Tierschutzbeauftragter) and all necessary licenses were obtained from the regional Ethical Commission for Animal Experimentation of Dresden, Germany (Tierversuchskommission, Landesdirektion Dresden).

Experiments were performed on 6-10 weeks old male C57BL/6JHsd mice (Harlan laboratories) and male or female Lifeact-EGFP mice (obtained from the laboratory of R. Wedlich-Soldner (Riedl et al., 2010)). For IVM of CF(DA) transport, animals were starved for 6 h (water ad libitum) prior experiments. For imaging of BC contractility in Lifeact-EGFP mice, animals were not starved. Fasudil and APAP were dissolved in 0.9% saline and administered at 20 mg/kg (Fasudil) or 150 mg/kg (APAP) by i.p. injection at 1.5 h (Fasudil) or 2 h (APAP) prior imaging or organ collection. As controls, mice were administered 0.9% saline.

Protein Extraction and Western Blot

Liver tissue was lysed in ice-cold 20 mM Tris-hydrochloride (Tris-HCl) pH 7.5, 150 mM sodium hydrochloride (NaCl), 1 mM ethylenediaminetetraacetic acid (EDTA), 1 mM ethylene glycol-bis(2-aminoethylether)-tetraacetic acid (EGTA), 1% (w/v) sodium dodecyl sulfate (SDS), 1% (w/v) NP-40 using a pestle. Cell debris were removed by centrifugation for 10 min at 12 000×g at 4° C. and protein was denatured at 95° C. in presence of 100 mM dithiothreitol (DTT). A total of 10 μg protein was separated by sodium dodecyl sulfate polyacrylamide gel electrophoresis, and transferred onto a nitrocellulose membrane.

Membranes were blocked and incubated with primary antibodies against glyceraldehyde 3-phosphate dehydrogenase (GAPDH, 1:2000) or phospho-myosin light chain (pMLC, 1:500) and HRP-conjugated secondary antibodies (1:10000) in 5% dry-milk, 10 mM Tris-HCl pH 8.0, 200 mM NaCl, 0.1% Tween20. Protein was detected using the enhanced chemiluminescence (ECL) detection kit (GE Healthcare, Buckinghamshire, UK) and chemiluminescence films (GE Healthcare, Buckinghamshire, UK) according to manufacturer's instructions.

Liver Tissue Fixation and Immunofluorescence Staining

Liver tissue of WT and Fasudil-treated animals was fixed by trans-cardial perfusion (3.7 ml/min) with 4% paraformaldehyde (PFA), 0.1% Tween in phosphate buffered saline (PBS) and post fixed in 4% PFA, 0.1% Tween, PBS (for immunofluorescence microscopy, IF) or in 2% glutaraldehyde, PBS (for electron microscopy, EM) at 4° C. overnight. Liver tissue of APAP-treated animals and controls were only immersion fixed in 4% PFA, 0.1% Tween20 in PBS for 48 h at 4° C. to avoid artefacts (trans-cardial perfusion caused BC network blebbing). For IF stainings, fixed liver tissue was mounted in 4% low melting agarose in PBS and sectioned into 100 μm thick slices using a vibratome (Leica VT1200S). Floating sections were permeabilized in 0.5% Triton-X100 in PBS for 1 h, quenched by incubation with 10 mM ammonium chloride (NH4Cl) in PBS for 30 min and blocked by incubation with blocking buffer (0.2% fish gelatin, 300 mM NaCl, 0.3% Triton-X100 in PBS) 3 times for 5 min. Sections were incubated sequentially with a primary antibody against CD13 (1:500) in blocking buffer for 2 overnights, washed 5 times for 5 min with 0.3% Triton-X100 in PBS, incubated with secondary antibody labelled with Alexa fluorophore 568 (1:1000), DAPI (1:2000) and Alexa fluorophore 488-conjugated Phalloidin (1:400) in blocking buffer for 2 overnights and washed again with 0.3% Triton-X100 in PBS 5 times for 5 min. Sections were optically cleared with SeeDB (See Deep Brain) and imaged using 80% (v/v) 2,2′-thiodiethanol as immersion medium as described previously (Ke et al., 2013). All steps were performed at room temperature.

Microscopy of Fixed Tissue

Fixed tissue was imaged with a Zeiss laser scanning microscope 780 NLO using a 63×1.3 numerical aperture (NA) glycerol immersion objective (Zeiss), a Chameleon Ti-Sapphire 2-photon laser (780 nm), 488 and 561 laser lines and Gallium arsenide phosphide (GaAsp) detectors.

Intravital Imaging of CFDA Transport

Mouse anaesthesia was induced with 3-4% isoflurane/0.3% oxygen and maintained by i.p. injection of ketamine/xylazine throughout the experiment. Mice received 20 U of heparin dissolved in 0.9% (w/v) NaCl by i.p. injection to prevent ischemia of the liver during the course of imaging. To visualize hepatocyte nuclei, Hoechst 33258 dissolved in 0.9% (w/v) NaCl was administered retro-orbitally at a dose of 2 mg/kg and in a total volume of 50 μl. To expose the liver for imaging, the abdominal fur was removed using an electric shaver and a small transversal incision of 1 cm was made at the height of the sternum to expose the left lateral liver lobe using a surgical scissor and cauterizer. The mouse was positioned on the stage of an IX81 inverted confocal microscope equipped with a Fluoview 1000 scanning head (Olympus America) and a heat-adjustable UPLSAPO30X, 30×1.05 NA silicon oil objective (Olympus). The objective was heated to 37° C. To avoid compression of the liver lobe and to stabilize the tissue, the stage was designed with a small hole in the center into which the left lateral lobe was carefully placed. The residual volume of the hole was filled with a water based and transparent 1% carbomer gel (0.3 M Sorbitol, 1% (w/v) Carbomer 940, polymerized by addition of triethanolamine and adjusted to pH 7) to immobilize the organ and prevent the organ from drying out. The bottom of the hole was designed with a coverslide through which the organ was accessible for imaging. The animal body temperature was kept at 37° C. using a red lamp. Prior to imaging start, 18 mg/kg of 70 kDa Rhodaminedextran or 20 μl of Qtracker 655 vascular label dissolved in 0.9% (w/v) NaCl was injected retro-orbitally to identify the lobule orientation from the vascular flow pattern. To monitor the flux of CFDA in the liver, CFDA, dissolved in dimethyl sulfoxide (DMSO), was injected retro-orbitally at a dose of 0.2 mg/kg in a total volume of 50 μl. CFDA injection was performed slowly (approx. 30 sec) to avoid hydrodynamic effects and performed at 1 min after image acquisition start. The imaging field covered an entire CV-PV axis in the first cell layers below the liver capsule. Movies were acquired at temporal resolution of 1 min (WT, APAP and APAP control) or 2 min (Fasudil and Fasudil control) over a time course of 1 h. For each time point a 20 μm stack with an image size of 320×320 μm, a pixel size of 0.5-1 μm and 1 μm z-steps was acquired.

Intravital Imaging of BC Contractility

For intravital imaging of BC contractility the mouse preparation was the same as described for imaging of CFDA except of the following steps: Anaesthesia of Lifeact-EGFP mice was maintained using 3-4% isoflurane/0.3% oxygen throughout the entire experiment and imaging was performed using a Leica-DMI6000 inverted microscope with a heated stage (37° C.). Images were acquired using a 8 kHz resonant galvo-scanner and a Leica HC CS2 PL APO 63×1.3 NA glycerol objective that was heated to 37° C. Lifeact-EGFP was excited using a 488 laser and detected using a hybrid detector. BC were imaged within the first 1-2 cell layers below the liver capsule. Elastin fibres of the liver capsule were imaged by SHG microscopy using a tuneable 2-photon laser at 900 nm and a non-descanned photomultiplier tubes (PMT) detector. Movies were acquired with a pixel size of 0.09 μm and 0.020 or 0.025 sec frame rates.

Electron Microscopy

1-2 mm³ pieces of fixed liver tissue were processed for SBF-SEM pre-embedding staining, and flat embedding on glass slides in hard Durcupan resin as described previously (Deerinck et al., 2010). Small pieces of embedded tissues were cut from the slides, remounted on SBFSEM pins using conductive glue and carbon coated. SBF-SEM was performed on a Magellan 400 SEM (Fei, The Netherlands), equipped with a Gatan 3ViewXP2 (Gatan, Munich) at 1.5 kV, 100 pA beam current, using a 12.4 nm pixel size and 40 nm-thick sections.

### Example 2

Mathematical and Computational Models

3D Reconstruction and Spatial Analysis of BC Network Geometry

3D reconstructions of the BC network were performed on high-resolution IF image stacks of fixed liver tissue stained for the apical marker CD13 (voxel size: 0.28×0.28×0.3 μm, 70-80 μm in depth). A tile of 2×1 image stacks was stitched to cover an entire CV-PV axis. Images were processed, analyzed and reconstructed using the software MotionTracking as described in (Morales-Navarrete et al., 2015). In brief, images were segmented using a local thresholding algorithm (maximum entropy), segmented objects were corrected for artefacts using standard morphological operations (opening/closing) and the triangulation mesh of the segmented surfaces was generated by the cube marching algorithm. The active mesh was tuned to align the triangle mesh vertexes to the maximum gradient of fluorescence intensity in the original image. A representation of the skeletonized image was generated using a 3D graph describing the geometrical and topological features of the BC network. For the spatial analysis of BC radius, porosity and network density, the CV-PV axis was computationally divided into 20 equidistant zones based on the distance dof each position x from the CV (dCV) and PV (dPV):

$$x = \frac{{d}_{CV}}{{d}_{CV} + {d}_{PV}}$$

Then, the average radius and porosity was determined per zone. For the radius, the minimal distance of each node of the central line of the BC network was quantified in the xy-plane. For the BC network density, the network length per tissue volume was calculated. For the tissue porosity,

$$ɛ = \frac{{V}_{V}}{{V}_{τ}}$$

the BC network was computationally divided into cubes of 20 μm³ that had a 5 μm grid spacing. For each cube, the porosity E was calculated as the ratio of the void tissue volume of the BC network V_{V} to total tissue volume V_{T}:

Then, the average porosity from all cubes within a zone was determined.

To determine the shortest BC network path between the PV and CV area, BC network endnodes were defined based on their distance to the CV and PV. For each end-node in the CV area the shortest distance through the BC network to an end-node in the PV area was calculated using using the Dijkstra's algorithm. From the calculated paths, the shortest one was chosen. To determine the direct CV-PV axis distance, the distance from each point of the CV to the closest one of the PV was calculated.

3D Reconstruction of a BC from EM Image Stacks

SBF-SEM images were de-noised by applying a median filter and aligned using Matlab. The images were segmented by intensity thresholding and size filtering using the Imaris software and the BC volume meshes were generated using the snappyHexMesh utility in openFoam (http://www.openfoam.org).

Correction and Quantification of IVM Movie Shift

Frames of CFDA transport movies were aligned by transitional image registration. The dextran or Hoechst channel was used as reference to align the frames of all other channels (target image) to it. The relative shift ([x,y] in 2D and [x,y,z] in 3D) of the target image stack was calculated using the phase correlation approach. The cross-correlation between stacks was computed using the Fast Fourier Transform. To determine the stability of the IVM setup, the same algorithms were applied to calculate the image shift in 2D on movies of elastin fibres.

Quantification of CF Intensities from Intravital Movies

Quantification of the CF intensity in the hepatocyte cytoplasm and BC from IVM movies was performed using the image analysis software MotionTracking. Following correction for shift, IVM movies contained 16-21 frames per stack, covering 15-20 μm in z. To avoid liver damage from photo-toxicity, IVM movies were acquired with minimum laser intensities resulting in low signal-to-noise ratio. The resulting difference of intensity between the background and the CF fluorescence in the hepatocyte cytosol was in the range of 5-20 intensity units and varied with depth. Therefore, we used the modified Mean-Shift algorithm to separate the CF fluorescence from the background. The result of the background/foreground discrimination was checked manually. The BC were detected in three steps. First, the image was convolved with Laplacian of Gaussian. Second, pixels with an intensity >2 standard deviations of the local noise were defined as potential BC. The local noise was determined from the local intensity minimums in at least one of 4 discrete directions. Third, along the line of the local maximum intensity direction of a BC, pixels that co-localized with sharp intensity transitions between the cytoplasm and sinusoids were excluded from the BC compartment. For all compartments (cytoplasm, BC and background), the mean intensity was calculated. To avoid mixing of compartments from light scattering of bright objects (BC to cytoplasm and cytoplasm to background), the area within the radius of 2 pixels from the object border was excluded from the calculation.

Following segmentation, the mean intensity of the BC and cytoplasm was corrected for the intensity of the background. Due to the low signal-to-noise ratio, the direct subtraction of background intensities from the cytosolic intensities sometimes resulted in negative values and consequently created artefacts. To avoid these artefacts, we developed a Bayesian estimation of the most probable value of the foreground intensity by considering the noise of the background and foreground intensity measurements. In short, we denoted f and b to be the foreground and background intensities which we want to estimate and m and n to be the foreground and background intensities we have measured. Then the probability of f, b given m, n is

$$p ( f , b \vert m , n ) = \frac{1}{Z ( m , n )} p ( m , n \vert f , b ) p ( f ) p ( b )$$

where p(f) and p(b) are prior distributions of the foreground and background.

We assumed that m, n are independent and normally distributed. This assumption is reasonable, because the intensities were estimated by the summation of thousands of pixels (see above) and according to the Central Limit Theorem these distributions have to converge to a normal one.

Therefore:

$$p ( m , n \vert f , b ) = \frac{1}{2 π {σ}_{1} {σ}_{2} mn} exp ( - \frac{1}{2} * ( \frac{{ln}^{2} ( \frac{f + b}{m} )}{{σ}_{1}^{2}} + \frac{{ln}^{2} ( \frac{b}{n} )}{{σ}_{2}^{2}} ) )$$

Since we were not interested in the background value, we marginalized it:

$$p ( f \vert m , n ) = \frac{p ( f )}{Z ( m , n )} {\int }_{0}^{∞} exp ( - \frac{1}{2} * ( \frac{{ln}^{2} ( \frac{f + b}{m} )}{{σ}_{1}^{2}} + \frac{{ln}^{2} ( \frac{b}{n} )}{{σ}_{2}^{2}} ) ) p ( b ) db$$

We assumed uniform improper prior for f and b: p(b)=const and p(f)=const that resulted in:

$$p ( f \vert m , n ) = \frac{1}{Z ( m , n )} exp ( - \frac{1}{2} \frac{{( m - n - f )}^{2}}{{σ}_{1}^{2} + {σ}_{2}^{2}} ) ( 1 + erf ( \frac{( m - f ) {σ}_{2}^{2} + n {σ}_{1}^{2}}{\sqrt{2} ( {σ}_{1}^{2} + {σ}_{2}^{2} ) S} ) )$$

where

$${S}^{2} = {( \frac{1}{{σ}_{1}^{2}} + \frac{1}{{σ}_{2}^{2}} )}^{- 1}$$

and Z(m,n) is normalization constant. The planes in a 3D stack were split on 16 sub-images and the values σ1 and σ2 were estimated from the variation of the intensity measurements between the sub-images within z-planes and across z-planes in a 3D image stack. The most probable value of f was found numerically as

$$f = {max}_{f ∈ ( 0 , ∞ )} \{ exp ( - \frac{1}{2} \frac{{( m - n - f )}^{2}}{{σ}_{1}^{2} + {σ}_{2}^{2}} ) ( 1 + erf ( \frac{( m - f ) {σ}_{2}^{2} + n {σ}_{1}^{2}}{\sqrt{2} ( {σ}_{1}^{2} + {σ}_{2}^{2} ) S} ) ) \}$$

Then, the resulting estimations of the compartment intensities were multiplied by their total pixel number to calculate the final integral intensity of the compartment.

Within an experimental condition (WT, controls, Fasudil or APAP-treated mice) intensities from each movie were scaled to the mean value of the condition by applying a scaling factor that was calculated as:

$${f}_{i} = \frac{{\sum }_{j = 1}^{N} {y}_{i , j} 〈 {y}_{j} 〉}{{\sum }_{j = 1}^{N} {y}_{i , j}^{2}}$$

Where y_{i,j} is an intensity of i-th curve in j-th time point, <yⱼ> is a mean intensity in j-th time point and fᵢ is a scaling factor for i-th curve. For each experimental condition, the mean intensity curve was calculated from 4-5 IVM movies and later used for further mathematical modelling.

IVM movies of Lifeact-EGFP were de-noised by applying a mean filter using the Fiji software. All other IF images and movies were intensity threshold adjusted but not processed otherwise.

Mathematical 3-Compartment Model of CF(DA) Transport in the Liver

The transport of CF(DA) from the blood into and through the biliary network was described in a mathematical model that considers 3 hepatic compartments: the blood (s), hepatocytes cytoplasm (c) and bile canaliculi (b) (see FIG. 2F). Dye propagation through these three compartments was modelled for three spatially distinct zones within the liver lobule: The CV, MD and PV zone as defined in FIG. 2C. The different zone geometries of the kite-shaped CV-PV axis were accounted for by introducing respective geometry factors. The model considers that only a fraction of the injected CFDA is delivered to the liver, whereas the rest is cleared by other tissues at rate κ_{clear}. The injection of CFDA into the blood circulation is described by Pulse(t). It is defined by the time point of injection start (τ1) and the injection duration (τ2) assuming that CFDA is injected with constant rate in time t>τ1 and <τ1+τ2. Both τ1 and τ2 were fitted by the model with boundary intervals 40-80 sec (τ1) and 20-60 sec (τ2). In the liver, the model considers that CFDA is taken up from the blood with a permeability coefficient Perm=3 sec⁻¹ (according to (Breeuwer et al., 1995), considering a mammalian plasma membrane thickness of 4 nm), converted into CF by cytoplasmic esterases with cleavage rate κ_{cleav} and secreted by apical transporters with transporter activity κₚᵤₘₚ. To account for the spatial heterogeneity of esterase and transporter levels within the CV-PV axis, the amounts of both esterases (qᵢ) and apical transporters (q1ᵢ) were determined for each zone (i=cv, md, pv). The model further considers passive back flux of CF from the BC into the hepatocyte cytoplasm at rate κₗ_{ᵢ}. The bile canaliculi compartment of the three zones is connected, occurs unidirectional and sequential from the CV to the MD with transport rate

$${k}_{{t}_{{cv}_{md}}}$$

and form the MD to the PV zone with rate

$${k}_{{t}_{{md}_{pv}}}$$

and exits the network from the PV zone into the bile duct with transport rate

$${k}_{{t}_{{pv}_{out}}} .$$

The dynamics of CF(DA) were described by a set of ODEs. In the blood compartment (Cₛ), the flux of the tracer was described as:

$$\frac{{dC}_{s}}{dt} = Perm * Pulse ( t ) - {k}_{clear} * {C}_{s}$$

The flux of the tracer was described separately for the cytoplasmic compartment (C_{c}_{ᵢ}) as:

$$\begin{matrix} \frac{{dC}_{{c}_{cv}}}{dt} = \frac{{V}_{cv} * {k}_{cleav} * {q}_{cv}}{( {q}_{cv} + {C}_{s} ) * {C}_{s}} + {k}_{{l}_{cv}} * {C}_{{b}_{cv}} - \frac{{k}_{pump} * q {1}_{cv}}{( q {1}_{cv} + {C}_{{c}_{cv} )} * {C}_{{c}_{cv}}} & ( Ia ) \\ \frac{{dC}_{{c}_{md}}}{dt} = \frac{{V}_{md} * {k}_{cleav} * {q}_{md}}{( {q}_{md} + {C}_{s} ) * {C}_{s}} + {k}_{{l}_{md}} * {C}_{{b}_{md}} - \frac{{k}_{pump} * q {1}_{md}}{( q {1}_{md} + {C}_{{c}_{md}} ) * {C}_{{c}_{md}}} & ( Ib ) \\ \frac{{dC}_{{c}_{pv}}}{dt} = \frac{{V}_{pv} * {k}_{cleav} * {q}_{pv}}{( {q}_{pv} + {C}_{s} ) * {C}_{s}} + {k}_{{l}_{pv}} * {C}_{{b}_{pv}} - \frac{{k}_{pump} * q {1}_{pv}}{( q {1}_{pv} + {C}_{{c}_{pv}} ) * {C}_{{c}_{pv}}} & ( Ic )\end{matrix}$$

And for the bile canaliculi compartment (C_{bi}) as:

$$\begin{matrix} \frac{{dC}_{{b}_{cv}}}{dt} = \frac{{k}_{pump} * q {1}_{cv}}{( q {1}_{cv} + {C}_{{c}_{cv}} ) * {C}_{{c}_{cv}}} - {k}_{{l}_{cv}} * {C}_{{b}_{cv}} - {k}_{{t}_{{cv}_{md}}} * {C}_{{b}_{cv}} & ( IIa ) \\ \frac{{dC}_{{b}_{md}}}{dt} = \frac{{k}_{pump} * q {1}_{md}}{( q {1}_{md} + {C}_{{c}_{md}} ) * {C}_{{c}_{md}}} - {k}_{{l}_{md}} * {C}_{{b}_{md}} + {k}_{{t}_{{cv}_{md}}} * {C}_{{b}_{cv}} - {k}_{{t}_{{md}_{pv}}} * {C}_{{b}_{md}} & ( IIb ) \\ \frac{{dC}_{{b}_{pv}}}{dt} = \frac{{k}_{pump} * q {1}_{pv}}{( q {1}_{pv} + {C}_{{c}_{pv}} ) * {C}_{{c}_{pv}}} - {k}_{{l}_{pv}} * {C}_{{b}_{pv}} + {k}_{{t}_{{md}_{pv}}} * {C}_{{b}_{md}} - {k}_{{t}_{{pv}_{out}}} * {C}_{{b}_{pv}} & ( IIc )\end{matrix}$$

All parameters were fitted to the experimental data by maximizing the Gaussian likelihood function using the simulation software FitModel (Zeigerer et al., 2012). Since the rate parameters could vary by orders of magnitude the logarithms of the rates were used as fitting parameters. The confidence intervals for parameters were estimated by numerical calculation of the inverse Hessian matrix of the Gaussian likelihood function (Sivia and Skilling, 2006).

Apical secretion rates sqi were calculated relative to the MD zone as follows:

$${sq}_{i} = \frac{q {1}_{i} * {v}_{md}}{q {1}_{md} * {v}_{i}}$$

Bile flow velocity vi at the interfaces of the lobule zones was estimated from the volume flux rates

$${k}_{{t}_{{cv}_{md}}} , {k}_{{t}_{{md}_{pv}}} and {k}_{{t}_{{pv}_{out}}}$$

considering the size of the zone volumes V and zone boundaries Aᵢ (i=cv, md, pv) as well as a CV-PV axis distance L of 229 μm:

$$\begin{matrix}{v}_{cv} = \frac{{k}_{{t}_{{cv}_{md}}} * {V}_{cv}}{{A}_{cv}} * L & ( IIIa ) \\ {v}_{md} = \frac{{k}_{{t}_{{md}_{pv}}} * {V}_{md}}{{A}_{md}} * L & ( IIIb ) \\ {v}_{pv} = \frac{{k}_{{t}_{{pv}_{out}}} * {V}_{pv}}{{A}_{pv}} * L & ( IIIc )\end{matrix}$$

Computational Fluid Dynamics Simulation of Bile Flow in a 3D EM-Reconstructed BC

The simulation of bile flow in a 3D reconstructed bile canaliculus from EM was carried out in the laminar regime as an incompressible and Newtonian fluid for physiologically relevant bile pressure conditions (10⁻³-10⁵ Pa) and in steady state mode using the open-source software openFoam (http://www.openfoam.org) and, specifically, the simpleFoam laminar solver therein. Grid convergence studies were conducted in the standard way in order to ascertain robustness and accuracy of the numerical simulation. To verify that bile flow inside BC is laminar, we calculated the Reynolds number (Re) from the orders of magnitude of our measured bile velocity and BC diameter as well as bile density and bile viscosity (Luo et al., 2007) and obtained

$$Re ∼ \frac{1 \frac{µm}{sec} * {10}^{3} \frac{kg}{{m}^{3}} * 1 µm}{1 mPa * sec} = {10}^{- 6}$$

which lies 9 orders of magnitude below the threshold to turbulent flow.

Mechanistic Model of Osmotic Fluid Secretion and Bile Flow

We developed a mechanistic model of osmotically driven secretion of biliary fluid to predict the spatial water influx profile j(x) into the BC network and the bile flow velocity vᵢ at the borders of zones i=cv, md, pv along the CV-PV axis (see FIG. 2C for the definition of the zones). The model takes into account previously reported fluid mechanic properties of bile (Luo et al., 2007) and the measured geometrical parameters, including the canaliculus radius profile a(x)=aₐ(x)*r_{corr} with apparent radius aₐ(x) (e.g. shown in FIG. 3C for WT mice) and correction factor r_{corr}=0.344 for the hydraulic diameter or radius, a mean cumulative BC path length L between the CV and PV of 344 μm and a linear coordinate x along a shortest path of BC branches from a tip of the BC network near the CV (x=0) to a junction with a draining bile duct in the PV area (x=L), neglecting potential effects of branch points (FIG. 4B). The osmotic driving due to osmolites with concentration c in the BC lumen is considered spatially uniform (see justification below) and static (independent of the CF pulse) pₒₛₘ=RTc, causing BC water influx j(x) to be proportional to the pressure surplus of pₒₛₘ over the local fluid pressure p(x). The local fluid pressure p(x) is monotonously decreasing from a self-organising pressure maximum at the closed central tip to zero at the open outlet, hence also j(x) is non-uniform in the general case. This feedback mechanism (FIG. 4B) was modelled by a first order ODE for the fluid velocity profile v(x) at steady state according to (Mathias, 1985):

where κ is the water permeability of the apical membrane of hepatocytes, the factor 2/a accounts for the surface to volume ratio and v(x) is the cross-section average of the local fluid velocity. We assumed Poiseuille flow with

$$v ( x ) = \frac{- {a}^{2}}{8 μ} \frac{dp}{dx}$$

where μ is the fluid viscosity of bile and applied mixed boundary conditions with zero inflow v(x=0)=0 at the closed central tip (corresponding to the Neumann boundary condition dp/dx=0 for pressure as a(x=0) is finite) and the Dirichlet condition p(x=L)=0 at the open outlet. The solution is

$$v ( x ) = {v}_{0} \sqrt{M} \frac{sinh ( \sqrt{M} x / L )}{cosh \sqrt{M}}$$

with two parameters, a velocity amplitude

$${v}_{0} = \frac{{a}^{2} RTc}{8 µL}$$

and the dimensionless Münch number

$$M = \frac{16 κ μ {L}^{2}}{{a}^{3}} .$$

To factor out the measured geometrical parameters, we rewrote

$${v}_{0} ( x ) = \frac{{a ( x )}^{2} {p}_{1}}{L} and M ( x ) = \frac{{p}_{2} {L}^{2}}{{a ( x )}^{3}}$$

with fit parameters p₁ and p₂. The heterogeneous radius profile a(x)=aₐ(x)*r_{corr} weakly modulated both terms such that the analytical solution remained a valid approximation of the heterogeneous problem. Matlab was used to fit p₁ and p₂ such that the analytical solution reproduced the measured bile flow velocity values at the border of each of the three zones (CV, MD, PV) and to determine the full spatial profile of the flow velocity v(x). The used approximation of a spatially uniform osmolite concentration profile is valid for the contributing small molecules and ions. These typically have diffusion constants of the order of D=100 μm²/sec. Even if continuously secreted only at one location, decay with a half-life of τ=20 min results in a steady state concentration profile with a decay length of \sqrt{Dτ}>L.

From the continuity equation we further calculated

$$j ( x ) = {j}_{0} ( x ) \frac{cosh ( \sqrt{M ( x )} x / L )}{cosh \sqrt{M ( x )}}$$

with j₀(x)=2πa(x)κpₒₛₘ. The resulting water influx profile was monotonously increasing (FIG. 4C).

Using the equation for v₀(x) from above to replace pₒₛₘ, we could express j₀ purely as a function of the geometrical parameters of the canaliculus and the parameters v₀(x) and M(x) of the velocity solution above and obtained:

$${j}_{0} ( x ) = \frac{π {a ( x )}^{2} {v}_{0} ( x ) M ( x )}{L}$$

Given the measured fluid velocities v, we could predict the complete fluid source density profile j(x) independent of the exact parameter values for membrane permeability κ, fluid viscosity μ or osmotic driving pₒₛₘ.

Inference of Osmolite Concentration Changes and Prediction of Parameter Changes

We considered the steady state balance of osmolites for the kite-shaped part of the lobule as a whole. Then the sum of sources from apical secretion,

J=j_{CV}+j_{MD}+j_{PV}

equals the sum of sinks from portal outflow

J=c*A_{duct}*v(x=L)

where A_{duct} is a fixed geometrical property related to the cumulative cross section area of all BC junctions with bile ducts. We considered the ratio of this equality for two conditions, denoted by subscripts control and treated for all quantities

$$\frac{{J}_{t}}{{J}_{c}} = \frac{{c}_{t} * {v}_{t} ( L )}{{c}_{c} * {v}_{c} ( L )}$$

We approximated the apical secretion fluxes of osmolites by the measured apical secretion fluxes of CF and obtained flux ratios

$$\frac{{J}_{t}}{{J}_{c}}$$

at t=500 sec when image segmentation had the highest accuracy due to near-maximum CF intensities in all compartments,

$$\frac{{J}_{Fasudil}}{{J}_{control}} = 0.464 and \frac{{J}_{APAP}}{{J}_{control}} = 0.798 .$$

Next, we considered the ratio of the analytical solutions (x) at x=L, that are proportional to pₒₛₘ=RTc, for each pair of conditions and calculated their ratio. From the two equations for

$$\frac{{J}_{t}}{{J}_{c}} and \frac{{v}_{t} ( L )}{{v}_{c} ( L )}$$

we then eliminated the velocity ratio

$$\frac{{v}_{t}}{{v}_{c}}$$

and obtained the osmolite concentration ratio

$$α = \frac{{c}_{t}}{{c}_{c}} = \sqrt{\frac{{J}_{t}}{{J}_{c}} * \frac{{{a}_{c} ( L )}^{2} \sqrt{{M}_{c} ( L )} tanh \sqrt{{M}_{c} ( L )}}{{{a}_{t} ( L )}^{2} \sqrt{{M}_{t} ( L )} tanh \sqrt{{M}_{t} ( L )}}}$$

as a dimensionless number a. Note that osmolite concentration only grows with the square root of the osmolite secretion flux as a result of the negative feedback by induced water secretion and resulting osmolite washout (see FIG. 4B). Therewith the alteration of parameter p₁ due to osmolite secretion changes compared to a control condition could be calculated

$${p}_{1 t} = \frac{RT {c}_{t}}{8 μ} = {p}_{1 c} * α$$

to then predict corresponding alterations in the velocity profile. Parameter p₂ₜ=p_{2c} remained unchanged as it is independent of geometric properties and the osmolite concentration.

To demonstrate the use of directly measured geometric parameters (apparent BC radii aₐ(x) for c(ontrol) and t(reated) conditions: a_{c}(x), aₜ(x)) and apical secretion fluxes J_{c}, Jₜ for determining

$${v}_{c} ( x ) = {p}_{1 c} \sqrt{{p}_{2 c} {r}_{corr} {a}_{c} ( x )} \frac{sinh ( \sqrt{\frac{{p}_{2 c}}{{r}_{corr}^{3} {{a}_{c} ( x )}^{3}}} x )}{cosh \sqrt{\frac{{p}_{2 c} {L}^{2}}{{r}_{corr}^{3} {{a}_{c} ( x )}^{3}}}}$$

parameter values and model predictions upon a change of treatment condition, we here explicitly consider the prediction of vₜ(x) upon Fasudil treatment given velocity measurements vᵢ for the control condition (i=cv, md, pv). First, the parameter values p_{1c} and p_{2c} for the control condition were determined by fitting the curve (just inserted the substitutions vₒ and M into the analytical model solution, see above)
to the experimental data points vᵢ in relative units. This yielded p_{1c}=566.9 μm⁻¹ and p_{2c}=6.656*10⁻⁶ μm which were confirmed by solving the initial ODE numerically using the software Morpheus (Starruss et al., 2014). Second, the apical secretion fluxes (in arbitrary intensity units per time) at t=500 sec were summed over the entire CV-PV axis: J_{c}=0.0041+0.0592+0.0209=0.0842 and Jₜ=0.0019+0.0312+0.0059=0.0390.

Third, using p₂ₜ=p_{2c}=6.656*10⁻⁶ μm and the extrapolated BC radii at x=L, a_{c}(L)=1.320 μm and aₜ(L)=1.422 μm, we calculated the osmolite concentration ratio (see above, after cancelling common factors)

$$α = \sqrt{\frac{{J}_{t}}{{J}_{c}} * \frac{\sqrt{{α}_{c} ( L )} tanh \sqrt{\frac{{p}_{2 c} {L}^{2}}{{r}_{corr}^{2} {{a}_{c} ( L )}^{3}}}}{\sqrt{{α}_{t} ( L )} tanh \sqrt{\frac{{p}_{2 t} {L}^{2}}{{r}_{corr}^{2} {{a}_{t} ( L )}^{3}}}}} = 0.6697 .$$

Fourth, the final parameter p₁ₜ=p_{1c}*α=379.7 μm⁻¹ was determined.

Inference of Peristaltic Contribution

We defined the contribution of peristalsis vₚ(x) to bile flow velocity as the difference between the measured velocity profile v_{c} (x) under control conditions (where osmosis and peristalsis coexist) and the velocity profile of osmotic driving alone vₒ(x) under the same condition. We obtained the velocity profile of sole osmotic driving under control conditions from the different condition of Fasudil treatment by first fitting our mechanistic model to the measured velocity profile v_{f}(x) upon Fasudil treatment and then calculating the corresponding velocity profile vₒ(x) under control conditions by inserting the measured radius profile a_{c}(x) and the predicted osmolite concentration, see also FIG. 5E. The resulting function (x)=(x)−(x) is displayed as solid black curve in FIG. 5F.

Next we computed the ratio

$$β ( x ) = \frac{{v}_{p} ( x )}{{v}_{c} ( x )}$$

to quantify the relative local contribution of peristalsis to total bile flow velocity, shown by the dashed black curve in FIG. 5F. For different experimental conditions with measured total bile flow velocity (x), we could then estimate the contribution of peristalsis as (x)=(x)*(x) and that of sole osmotic driving as (x)=(1−β/(x))*vt (x). Fitting the latter with our mechanistic model, we could predict the corresponding water influx density (x). We applied this workflow independently to the control of APAP treatment and the WT velocity profile. From the latter, we used the predicted fluid source profile in the 3D porous medium model.

From v_{APAPcontrol}_{ₒ}(X) we predicted v_{APAPtreated}_{ₒ}(x) using the measured radius profile and then the peristalsis contribution by

$${v}_{{APAPtreated}_{p}} ( x ) = \frac{β ( x )}{( 1 - β ( x ) )} * {v}_{{APAPtreated}_{o}} ( x )$$

$${v}_{APAPtreated} ( x ) = {v}_{{APAPtreated}_{o}} ( x ) + {v}_{{APAPtreated}_{p}} ( x ) = \frac{1}{( 1 - β ( x ) )} * {v}_{{APAPtreated}_{o}} ( x )$$

such that

A 3D Anisotropic Porous Medium Model of Biliary Fluid Dynamics

The liver lobule was considered as a regular hexagonal prism, with the z-axis along its center. The top and bottom hexagonal planes were characterized by a translational periodic boundary condition. The BC network was considered to transport bile from the center radially to the bile ducts located at the six corners of the hexagon. The network is closed at the central tips surrounding the CV (CV diameter=90 μm), but has open outlets at the portal triad (portal triad diameter=60 μm). Based on measurements from the 3D geometric model of the BC network, the distance from the surface of the CV to the portal triad is 229 μm. The hexagonal edges (representing the interfaces between lobules) and the center of the lobule (surrounding the central vein) were treated as bile-impermeable solid walls. The length of the z-axis was 269 μm. The lobule was considered as porous medium with a location-dependent porosity ϵ(x) along the CV-PV axis as determined by our geometric measurements (FIG. 3C, right y-axis). The porosity profile was described by a 10^{th} order polynomial fit of the experimental measurements. From the porosity profile, the location-dependent tissue permeability k(x) was estimated using the Kozeny-Carman model

k=(r*r_{corr})²*ϵ³

where r is the mean BC network radius of 1.143 μm (r=<a(x)>) determined from the geometric model, see also FIG. 3C) and r_{corr} is the correction factor to account for the hydraulic diameter of BC (see above). The spatial heterogeneity of the BC radius does not enter here since the heterogeneous geometry of the network was already accounted for by considering an anisotropic location-dependent porosity.

For the simulation, an average bile pressure of 100 Pa was applied at the bile duct (estimated according to measurements reported in (Wiener et al., 2000)) and bile was assumed to have a viscosity of 1 mPa*sec (Luo et al., 2007). Bile flow was considered to be driven by the osmotic effects of bile secretion and peristalsis. To simulate the osmotic effect, the local bile mass production rate (in units (kg*sec)/m³) was determined. Therefore, an empirical fit of the heterogeneous fluid source profile jₜ_{ₒ}(x) (in units μm³*sec/μm), predicted from the combined model of osmotic fluid secretion and peristalsis, was multiplied by the spatial BC branch density profile (the bile canaliculi network length per unit of tissue volume) (in units μm/μm³) and the bile density (928 kg/m³ (Mcintosh and Anderson, 2010)). To simulate fluid dynamics, the laminar Navier-Stokes equations at steady-state were numerically solved using the software ANSYS CFX (Ansys, Canonsburg, Pa., USA). The liver lobule geometry was meshed using the ICEM CFD software (Ansys, Canonsburg, Pa., USA). A mesh sensitivity study was carried out in order to define a mesh independent numerical solution. For that reason several grid configurations were used, resulting in 4.2 million tetrahedral elements. The total bile mass source in the liver lobule was estimated to be 9.784*10⁻¹¹ kg/sec.

To account for the additional contribution to bile flow velocity from BC network peristalsis, the bile velocity profile predicted by the porous medium model from the osmotic effects was multiplied by the space dependent factor 1/(1−β(x)), with β(x) representing the inferred relative contribution of peristalsis (see above). Bile velocity and pressure were normalized to the maximum values.

Statistical Analysis

In all experiments, n represents the number of mice used (see Figure legends for explicit n used per experiment). Intensity measurements of CF in the BC and hepatocyte cytoplasm from IVM movies of CFDA (FIG. 2D, E) are given as mean±68% confidence interval (CI). The uncertainty of transport parameters from the 3-compartment model are represented as 95% CI (FIG. 2G, FIG. 4C, FIG. 5E, FIG. 7D). It was estimated by calculation of the inverse Hessian matrix of the Gaussian likelihood function (Sivia and Skilling, 2006). Measurements of the BC radius, porosity and branch density from 3D reconstructions of confocal image stacks from WT, control, Fasudil-treated or APAP-treated mice (FIG. 3C, D, FIG. 5C, FIG. 7B) represent the mean±standard error of the mean (SEM) for each zone. The uncertainty of the inferred peristaltic contribution to the local bile velocity (FIG. 5F) and the prediction of bile velocity in control or APAP-treated mice (FIG. 7D) was obtained by propagation of the SEMs of the measured BC radius profiles shown in FIG. 5C and FIG. 7B, respectively. In FIG. 7D and S5G, the propagated uncertainty was then extrapolated across the CV-PV axis.

The significance for the difference between the model prediction of control from Fasudil vs. the experimental measure in control mice and for the difference between the model prediction of Fasudil from control vs. the experimental measure of Fasudil-treated mice (FIG. 5E) was estimated using the complementary error function. The significance of the peristaltic contribution to local bile velocity in the CV vs. PV zone was estimated by Student's t-test (FIG. 5F). The significance of the differences between the radius of control and Fasudil or APAP-treated mice (FIGS. 5C and 7B) was estimated by Student's t-test. The average pvalue for each zone comparing the difference between experimental measurements from APAP mice vs. model prediction of APAP from control (FIG. 7D) was calculated using the complementary error function.

### Example 3

Bile Canaliculi Contractility is a Determinant of Bile Flow

Earlier studies have provided evidence for BC contractility in the rat liver in vivo at a speed of less than one micron per second, suggesting a function for bile flow (Oshio and Phillips, 1981; Watanabe et al., 1991). Given the low viscosity of bile, one would expect a much higher speed of the peristaltic waves if these were to act as a driving force of bile flow. Since, to our knowledge, no further studies have corroborated the contractile activity of the BC in vivo and provided evidence that it significantly contributes to bile flow, we set out to test this hypothesis.

To verify that the biliary network has indeed contractile activity in vivo, we monitored the dynamics of BC geometry by IVM of Lifeact-EGFP mice using the subapical actin mesh as BC marker (Riedl et al., 2010). From cell culture studies, contractility of hepatocyte apical membranes has been shown to act in a calcium-dependent manner and, thus, suggested to be driven by calcium waves in vivo (Nathanson and Schlosser, 1996; Watanabe and Phillips, 1984; Watanabe et al., 1988). Given that calcium waves propagate at about 30 μm/sec across the lobule (Nathanson et al., 1995), BC peristalsis should occur at a similar speed, rendering its detection technically challenging. We therefore expected imaging at 0.025 sec frame rate and 0.09 μm pixel size to be sufficient to capture only individual BC constriction events of a peristaltic wave. Using these settings, we could indeed visualize contractile events occurring within a millisecond time frame (FIG. 5A). To rule out motion artefacts, we determined the image shift of our IVM setup. Using second harmonic generation (SHG) imaging of elastin fibres of the liver capsule as stable reference structure, we found the shift to be ≤0.27 μm in xy over a time course of 0.5 min, showing that contractility could be reliably measured for large BCs (diameter ≥2 μm). Thus, in contrast to a previous study, we found contractile events within the velocity range expected from the speed of calcium waves in the liver.

Plasma membrane contractility is determined by the cortical acto-myosin system (Sharanek et al., 2016; Tsukada and Phillips, 1993). Compromised acto-myosin activity has been suggested as a candidate mechanism of cholestasis-inducing drugs (Sharanek et al., 2016). Consequently, inhibition of acto-myosin contractility should affect biliary fluid dynamics. To test this prediction, we inhibited acto-myosin activity pharmacologically using the Rho kinase inhibitor Fasudil. Administration of 20 mg/kg Fasudil for 1.5 h resulted in a reduction of phosphorylated myosin light chain (pMLC) protein levels, thus validating the inhibitory effect on Rho kinase activity. We next analyzed the BC network geometry on fixed liver tissue sections and found a dilation of BC in Fasudil-treated mice (FIG. 5B). Quantification of BC radius revealed that Rho kinase inhibition dilated BC (FIG. 5C). The radius was significantly increased by up to 12% in the MD (pᵥₐₗᵤₑ<0.0001) and 7% in the PV zone (pᵥₐₗᵤₑ<0.001) as compared to control mice. This indicates that the diameter of the BC network is regulated by the acto-myosin system.

Finally, to determine the requirement of BC contractility for bile flow, we tested the effect of Rho kinase inhibition on bile velocity by IVM of CF(DA) transport (step 5 in FIG. 1). IVM of the BC network in Fasudil-treated mice revealed high sensitivity to photo-toxicity and was only possible at minimum laser intensities and lower time intervals (2 min) as compared to wild type (WT) mice. Imaging of CF transport in Fasudil-treated mice revealed that inhibition of myosin activity resulted in a marked delay of CF clearance from the BC network (FIG. 5D). We quantified a 50-58% reduction of bile velocity across the CV-PV axis upon acto-myosin inhibition as compared to control mice (FIG. 5E). Unexpectedly, Fasudil treatment also caused a reduction of apical CF transport.

Using the mechanistic model of bile secretion and flow, we could now determine whether the osmotic effect is sufficient to explain the measured reduction of bile velocity upon perturbation by Fasudil. Importantly, we took into account both the dilation of the BC and the reduction of apical osmolite (CF) transport upon Fasudil treatment. First, we determined the two parameters p1 and p2 of the osmotic model. To this end, we used the measured CV-PV axis distance L and the radius a(x) of control mice to fit p₁ and p₂ to the experimental data under control conditions (FIG. 5E, solid black line). Keeping the axis distance L and p₂ constant, and using the experimentally measured BC radius a(x) of Fasudil-treated mice, we then predicted the bile velocity profile under Fasudil treatment (FIG. 5E, dashed black line). The value of p₁ was corrected to account for the change of osmolite secretion upon Fasudil treatment (see Example 2, section ‘Inference of osmolite concentration changes and prediction of parameter changes’). The same strategy was applied to fit the osmotic model parameters p₁ and p₂ to the experimental data under Fasudil treatment (FIG. 5E, solid grey line) and predict the bile velocity profile under control conditions (FIG. 5E, dashed grey line; see also Example 2, section ‘Inference of peristaltic contribution’). Both predictions deviated from the experimental measurements by 45% in the PV zone. Despite considerable experimental variability in control mice, the difference between the model prediction and measured value under Fasudil treatment in the PV zone is highly significant (pᵥₐₗᵤₑ<0.0001). Therefore, by proof by contradiction, we concluded that the contribution of peristalsis has a significant effect for bile flow.

Using the model of the invention, we estimated the contribution of peristalsis to biliary flow (FIG. 5F). We defined the contribution of peristalsis to bile flow as the difference between the measured velocity profile under control conditions (where osmosis and peristalsis coexist, FIG. 5E, solid black curve) and the velocity profile of osmotic driving alone. The latter was estimated in two steps. First, we fitted the parameters of the osmotic model to the measurements of bile velocity of Fasudil-treated mice. Second, we used these parameters with the BC radius profile and apical secretion rates under control conditions to predict the velocity profile of sole osmotic driving (FIG. 5E, grey dashed line; see Example 2, section ‘Inference of peristaltic contribution’). The results suggest that peristalsis occurs particularly in the MD and PV zones, contributing up to 32%±3 (mean±SD) of the total bile velocity in the PV zone (FIG. 5F, black solid line). Considering the relative local contribution to velocity, peristalsis even accounts for 88%±6 (mean±SD) of the velocity in the CV zone (FIG. 5F, black dashed line). The local contribution is significantly higher in the CV than in the PV zone (pᵥₐₗᵤₑ<0.0001). This result could explain the underestimation of the measured bile velocity in the CV zone by the osmotic model (see FIG. 4C). Next, we determined the relative contribution of peristalsis to bile pressure and found a monotonously decreasing spatial profile along the CV-PV axis (FIG. 5F, grey line). Altogether, our results reveal that, in addition to the well-established osmosis-dependent mechanism, acto-myosin-driven contractility of the BC network is a key determinant of bile flow.

### Example 4

Bile Velocity and Pressure Establish Two Opposing Gradients in the Liver Lobule

In the liver lobule, bile flow does not occur through a single linear tube but through a highly ramified 3D network. To consider this geometry, we integrated structural and fluid dynamic properties from the (sub)-cellular level into a 3D lobule-scale model of bile flux using a porous medium approach (step 6 in FIG. 1). A porous medium approach models BC network topology as effective tissue porosity and permeability. We used our geometric model of the BC network to determine the spatial porosity profile within the CV-PV axis. Similar to the BC radius, the porosity profile revealed substantial heterogeneities within the lobule (FIG. 3C, right y-axis). Porosity specifically increased 2-fold in the second cell layer next to the CV and PV (maximum: 0.088±0.003, mean±SEM) compared to the middle area (minimum: 0.041+0.005, mean±SEM), indicating changes of network density within the lobule. We modelled such heterogeneity using an anisotropic permeability tensor which was estimated in a spatially resolved manner (see Example 2, section ‘A 3D anisotropic porous medium model of biliary fluid dynamics’).

Bile velocity and pressure within the porous medium were simulated by progressive integration of the osmotic and peristaltic effects on bile flow. First, we calculated the spatial profile of the bile production rate from the predicted profile of water influx into the BC network. To this end, we corrected our earlier model of osmotic bile secretion for the contribution of peristalsis. Second, we applied the spatial profile of the peristaltic contribution to bile velocity. Considering a bile pressure of 100 Pa at the bile ducts (Wiener et al., 2000), a CV-PV axis distance of 229 m and impermeable interfaces between lobules, the porous medium model predicted two inversely related gradients of bile velocity and pressure within the lobule (FIG. 6A, B).

Velocity streamlines followed the kite-shaped geometry of the CV-PV axis. The bile velocity increased gradually from the CV and along the MD zone (0-3.4 μm/sec) and rapidly accelerated in close proximity to the bile ducts (max. velocity 12 μm/sec) (FIG. 6A). In contrast to velocity, bile pressure decreased 30-fold from the CV to the PV area and dropped particularly fast near the bile ducts (FIG. 6B). The maximal bile pressure was predicted to reach 2474 Pa (18.6 mmHg, ca. 20% of blood pressure) in the CV area. Such a value is consistent with the reported maximum biliary secretion pressure of 1000-1500 Pa in the extrahepatic biliary system in rats (Weis and Barth, 1978). Taken together, both bile velocity and pressure change by about one order of magnitude along the CV-PV axis of the liver lobule.

### Example 5

A Sub-Toxic Dose of Acetaminophen Affect Biliary Fluid Dynamics

Our multi-scale model of biliary fluid dynamics is a promising tool to test and predict DILI of pharmacological compounds. Due to its clinical relevance, we chose the commonly used analgesic acetaminophen (APAP) to test it. APAP is metabolized in the liver and safe at therapeutic doses, but generates reactive metabolites upon overdose (>150 mg/kg) that can induce cholestasis from toxic hepatitis (Gillette et al., 1981; Potter and Hinson, 1987; Ruepp et al., 2002; Whitehouse et al., 1977). The reactive metabolites bind cytoplasmic proteins and, amongst others, cause BC network dilation and hepatocyte necrosis (Walker et al., 1983). Using our imaging and fluid dynamics modelling pipeline, we measured the effects of APAP on biliary flow properties. To exclude side-effects from hepatocyte necrosis, we chose the maximum reported sub-toxic dose of APAP (150 mg/kg) and analyzed the liver tissue 2 h after intraperitoneal (i.p.) administration. We verified that 150 mg/kg APAP did not cause significant toxic effects, such as cell death, as determined by DAPI and F-actin staining of liver tissue sections. With the exception of one sample that revealed peri-central BC network blebbing (area was excluded from the analysis), we found that APAP induced a significant dilation of BC by up to 20% in the CV (pᵥₐₗᵤₑ<0.01) and 18% in the MD zone (pᵥₐₗᵤₑ<0.0001) (FIG. 7A, B). Such effect of sub-toxic doses was surprising but consistent with the well-described spatial restriction of APAP toxicity to CV hepatocytes and previous reports on BC dilation upon APAP overdose (Walker et al., 1983).

Next, we tested our mechanistic model of osmotic fluid secretion and contractility by predicting bile flow upon APAP treatment. We first measured bile velocity by IVM of CF(DA) transport in control mice (FIGS. 7C and D) to calibrate our model assuming fixed relative contributions of osmotic effects and peristalsis (FIG. 7D, black line). Next, we predicted bile velocity from the measured radius profile upon APAP treatment (FIG. 7D grey line, see also Example 2, section ‘Inference of peristaltic contribution’). The model estimated up to 2.1-fold increase in bile velocity in the CV and MD zone. Consistent with this prediction, visualization and measurement of bile velocity from IVM of CF(DA) transport revealed a specific 1.4-1.6-fold increase in bile velocity in the CV and MD zones whereas the peri-portal zone remained almost unaffected (FIG. 7C, D). The difference between predicted and measured bile velocity upon APAP treatment was insignificant for all three zones (pᵥₐₗᵤₑ≥0.22). This is an independent test of the predictive power of our model of osmotic fluid secretion and peristalsis. In contrast to Fasudil, the effects of APAP on osmolite secretion were marginal. Consequently, changes of apical osmolite secretion upon APAP treatment had only a minor effect on the model results. This suggested that the observed increase in bile velocity upon APAP treatment is primarily caused by the BC network dilation in the CV and MD zones. Collectively, our results demonstrate the accuracy and applicability of our model of biliary fluid dynamics for pharmacological studies.

### FURTHER REFERENCES

Akita, H., Suzuki, H., Ito, K., Kinoshita, S., Sato, N., Takikawa, H., and Sugiyama, Y. (2001). Characterization of bile acid transport mediated by multidrug resistance associated protein 2 and bile salt export pump. Biochimica et biophysica acta 1511, 7-16.
Babbey, C. M., Ryan, J. C., Gill, E. M., Ghabril, M. S., Burch, C. R., Paulman, A., and Dunn, K. W. (2012). Quantitative intravital microscopy of hepatic transport. Intravital 1.
Baier, P. K., Hempel, S., Waldvogel, B., and Baumgartner, U. (2006). Zonation of hepatic bile salt transporters. Digestive diseases and sciences 51, 587-593.
Balistreri, W. F., Bezerra, J. A., Jansen, P., Karpen, S. J., Shneider, B. L., and Suchy, F. J. (2005).
Ballatori, N., and Truong, A. T. (1989). Relation between biliary glutathione excretion and bile acidindependent bile flow. The American journal of physiology 256, G22-30.
Ballatori, N., and Truong, A. T. (1992). Glutathione as a primary osmotic driving force in hepatic bile formation. The American journal of physiology 263, G617-624.
Baumgartner, U., Miyai, K., and Hardison, W. G. (1986). Greater taurodeoxycholate biotransformation during backward perfusion of rat liver. The American journal of physiology 251, G431-435.
Baumgartner, U., Miyai, K., and Hardison, W. G. (1987). Modulation of hepatic biotransformation and biliary excretion of bile acid by age and sinusoidal bile acid load. The American journal of physiology 252, G114-119.
Benhamouche, S., Decaens, T., Godard, C., Chambrey, R., Rickman, D. S., Moinard, C., Vasseur-Cognet, M., Kuo, C. J., Kahn, A., Perret, C., et al. (2006). Apc tumor suppressor gene is the “zonationkeeper” of mouse liver. Developmental cell 10, 759-770.
Berkowitz, C. M., Shen, C. S., Bilir, B. M., Guibert, E., and Gumucio, J. J. (1995). Different hepatocytes express the cholesterol 7 alpha-hydroxylase gene during its circadian modulation in vivo. Hepatology 21, 1658-1667.
Boyer, J. L. (2013). Bile formation and secretion. Comprehensive Physiology 3, 1035-1078.
Boyer, J. L., and Bloomer, J. R. (1974). Canalicular bile secretion in man. Studies utilizing the biliary clearance of (14C)mannitol. The Journal of clinical investigation 54, 773-781.
Boyer, J. L., and Klatskin, G. (1970). Canalicular bile flow and bile secretory pressure. Evidence for a non-bile salt dependent fraction in the isolated perfused rat liver. Gastroenterology 59, 853-859.
Breeuwer, P., Drocourt, J. L., Bunschoten, N., Zwietering, M. H., Rombouts, F. M., and Abee, T. (1995). Characterization of uptake and hydrolysis of fluorescein diacetate and carboxyfluorescein diacetate by intracellular esterases in Saccharomyces cerevisiae, which result in accumulation of fluorescent product. Applied and environmental microbiology 61, 1614-1619.
Coleman, R., lqbal, S., Godfrey, P. P., and Billington, D. (1979). Membranes and bile formation. Composition of several mammalian biles and their membrane-damaging properties. The Biochemical journal 178, 201-208.
Deerinck, T. J., Bushong, E. A., Lev-Ram, V., Shu, X., Tsien, R. Y., and Ellisman, M. H. (2010). Enhancing serial block-face scanning electron microscopy to enable high resolution 3-D nanohistology of cells and tissues. Microscopy and Microanalysis 16.
Elias, H. (1949). A re-examination of the structure of the mammalian liver; the hepatic lobule and its relation to the vascular and biliary systems. The American journal of anatomy 85, 379-456, 315 μl.
Gillette, J. R., Nelson, S. D., Mulder, G. J., Jollow, D. J., Mitchell, J. R., Pohl, L. R., and Hinson, J. A. (1981). Formation of chemically reactive metabolites of phenacetin and acetaminophen. Advances in experimental medicine and biology 136 Pt B, 931-950.
Giri, S., Nieber, K., and Bader, A. (2010). Hepatotoxicity and hepatic metabolism of available drugs: current problems and possible solutions in preclinical stages. Expert opinion on drug metabolism & toxicology 6, 895-917.
Groothuis, G. M., Hardonk, M. J., Keulemans, K. P., Nieuwenhuis, P., and Meijer, D. K. (1982). Autoradiographic and kinetic demonstration of acinar heterogeneity of taurocholate transport. The American journal of physiology 243, G455-462.
Jungermann, K. (1988). Metabolic zonation of liver parenchyma. Seminars in liver disease 8, 329-341.
Jungermann, K., and Kietzmann, T. (1996). Zonation of parenchymal and nonparenchymal metabolism in liver. Annual review of nutrition 16, 179-203.
Ke, M. T., Fujimoto, S., and Imai, T. (2013). See DB: a simple and morphology-preserving optical clearing agent for neuronal circuit reconstruction. Nature neuroscience 16, 1154-1161.
Layden, T. J., and Boyer, J. L. (1978). Influence of bile acids on bile canalicular membrane morphology and the lobular gradient in canalicular size. Laboratory investigation; a journal of technical methods and pathology 39, 110-119.
Liu, Y., Chen, H. C., Yang, S. M., Sun, T. L., Lo, W., Chiou, L. L., Huang, G. T., Dong, C. Y., and Lee, H. S. (2007). Visualization of hepatobiliary excretory function by intravital multiphoton microscopy. Journal of biomedical optics 12, 014014.
Luo, X., Li, W., Bird, N., Chin, S. B., Hill, N. A., and Johnson, A. G. (2007). On the mechanical behavior of the human biliary system. World journal of gastroenterology 13, 1384-1392.
Mathias, R. T. (1985). Epithelial water transport in a balanced gradient system. Biophysical journal 47, 823-836.
Mcintosh, R. L., and Anderson, V. (2010). A comprehensive tissue properties database provided for the thermal assessment of a human at rest. Biophys Rev Lett 05.
Morales-Navarrete, H., Segovia-Miranda, F., Klukowski, P., Meyer, K., Nonaka, H., Marsico, G., Chernykh, M., Kalaidzidis, A., Zerial, M., and Kalaidzidis, Y. (2015). A versatile pipeline for the multi-scale digital reconstruction and quantitative analysis of 3D tissue architecture. eLife 4.
Nathanson, M. H., Burgstahler, A. D., Mennone, A., Fallon, M. B., Gonzalez, C. B., and Saez, J. C. (1995). Ca2+ waves are organized among hepatocytes in the intact organ. The American journal of physiology 269, G167-171.
Nathanson, M. H., and Schlosser, S. F. (1996). Calcium signaling mechanisms in liver in health and disease. Progress in liver diseases 14, 1-27.
Oshio, C., and Phillips, M. J. (1981). Contractility of bile canaliculi: implications for liver function. Science 212, 1041-1042.
Padda, M. S., Sanchez, M., Akhtar, A. J., and Boyer, J. L. (2011). Drug-induced cholestasis. Hepatology 53, 1377-1387.
Paulusma, C. C., van Geer, M. A., Evers, R., Heijn, M., Ottenhoff, R., Borst, P., and Oude Elferink, R. P. (1999). Canalicular multispecific organic anion transporter/multidrug resistance protein 2 mediates low-affinity transport of reduced glutathione. The Biochemical journal 338 (Pt 2), 393-401.
Porat-Shliom, N., Tietgens, A. J., Van Itallie, C. M., Vitale-Cross, L., Jarnik, M., Harding, O., Anderson, J. M., Gutkind, J. S., Weigert, R., and Arias, I. M. (2016). Liver Kinase B1 regulates hepatocellular tight junction distribution and function in vivo. Hepatology.
Potter, D. W., and Hinson, J. A. (1987). Mechanisms of acetaminophen oxidation to N-acetyl-Pbenzoquinone imine by horseradish peroxidase and cytochrome P-450. The Journal of biological chemistry 262, 966-973.
Riedl, J., Flynn, K. C., Raducanu, A., Gartner, F., Beck, G., Bosl, M., Bradke, F., Massberg, S., Aszodi, A., Sixt, M., et al. (2010). Lifeact mice for studying F-actin dynamics. Nature methods 7, 168-169.
Ruepp, S. U., Tonge, R. P., Shaw, J., Wallis, N., and Pognan, F. (2002). Genomics and proteomics analysis of acetaminophen toxicity in mouse liver. Toxicological sciences: an official journal of the Society of Toxicology 65, 135-150.
Sharanek, A., Burban, A., Burbank, M., Le Guevel, R., Li, R., Guillouzo, A., and Guguen-Guillouzo, C. (2016). Rho-kinase/myosin light chain kinase pathway plays a key role in the impairment of bile canaliculi dynamics induced by cholestatic drugs. Scientific reports 6, 24709.
Sivia, D. S., and Skilling, J. (2006). Data Analysis—A Bayesian Tutorial. Oxford University Press, NY, 61-65.
Starruss, J., de Back, W., Brusch, L., and Deutsch, A. (2014). Morpheus: a user-friendly modeling environment for multiscale and multicellular systems biology. Bioinformatics 30, 1331-1332.
Trauner, M., Meier, P. J., and Boyer, J. L. (1998). Molecular pathogenesis of cholestasis. The New England journal of medicine 339, 1217-1227.
Tsukada, N., and Phillips, M. J. (1993). Bile canalicular contraction is coincident with reorganization of pericanalicular filaments and co-localization of actin and myosin-II. The journal of histochemistry and cytochemistry: official journal of the Histochemistry Society 41, 353-363.
Walker, R. M., Racz, W. J., and McElligott, T. F. (1983). Scanning electron microscopic examination of acetaminophen-induced hepatotoxicity and congestion in mice. The American journal of pathology 113, 321-330.
Watanabe, N., Tsukada, N., Smith, C. R., and Phillips, M. J. (1991). Motility of bile canaliculi in the living animal: implications for bile flow. The Journal of cell biology 113, 1069-1080.
Watanabe, S., and Phillips, M. J. (1984). Ca2+ causes active contraction of bile canaliculi: direct evidence from microinjection studies. Proceedings of the National Academy of Sciences of the United States of America 81, 6164-6168.
Watanabe, S., Tomono, M., Takeuchi, M., Kitamura, T., Hirose, M., Miyazaki, A., and Namihisa, T. (1988). Bile canalicular contraction in the isolated hepatocyte doublet is related to an increase in cytosolic free calcium ion concentration. Liver 8, 178-183.
Weis, E. E., and Barth, C. A. (1978). The extracorporeal bile duct: a new model for determination of bile flow and bile composition in the intact rat. Journal of lipid research 19, 856-862.
Whitehouse, L. W., Paul, C. J., Wong, L. T., and Thomas, B. H. (1977). Effect of aspirin on a subtoxic dose of 14C-acetaminophen in mice. Journal of pharmaceutical sciences 66, 1399-1403.
Wiener, S. M., Hoyt, R. F., Jr., Deleonardis, J. R., Clevenger, R. R., Jeffries, K. R., Nagashima, K., Mandel, M., Owens, J., Eckhaus, M., Lutz, R. J., et al. (2000). Manometric changes during retrograde biliary infusion in mice. American journal of physiology Gastrointestinal and liver physiology 279, G49-66.
Wielandt, A. M., Vollrath, V., Manzano, M., Miranda, S., Accatino, L., and Chianale, J. (1999). Induction of the multispecific organic anion transporter (cMoat/mrp2) gene and biliary glutathione secretion by the herbicide 2,4,5-trichlorophenoxyacetic acid in the mouse liver. The Biochemical journal 341 (Pt 1), 105-111.
Zamek-Gliszczynski, M. J., Xiong, H., Patel, N. J., Turncliff, R. Z., Pollack, G. M., and Brouwer, K. L. (2003). Pharmacokinetics of 5 (and 6)-carboxy-2′, 7′-dichlorofluorescein and its diacetate promoiety in the liver. The Journal of pharmacology and experimental therapeutics 304, 801-809.
Zeigerer, A., Gilleron, J., Bogorad, R. L., Marsico, G., Nonaka, H., Seifert, S., Epstein-Barash, H., Kuchimanchi, S., Peng, C. G., Ruda, V. M., et al. (2012). Rab5 is necessary for the biogenesis of the endolysosomal system in vivo. Nature 485, 465-470.

1. A computer-based method of predicting bile flow through a lobule of a mammalian liver, said method comprising:
(a) dividing the axis connecting the central vein and a portal vein of said lobule into a central zone, a middle zone and a portal zone, preferably based on the positions of said central vein and said portal vein as determined from microscopic images;
(b) measuring experimentally secretion of bile by hepatocytes;
(c) calculating the transport rate of said bile of (b) through each of the zones defined in (a), preferably using ordinary differential equations;
(d) providing a three-dimensional representation of the bile canaliculi in said lobule;
(e) calculating a first correction factor as the ratio between hydraulic radius and geometric radius of said bile canaliculi; and
(f) calculating bile transport through
(f-i) said three-dimensional representation of (d) using the transport rates determined in step (c) and said first correction factor calculated in step (e), preferably by solving the Navier-Stokes equations for said three-dimensional representation of (d); or
(f-ii) a three-dimensional porous medium model of said liver lobule using the transport rates determined in step (c);
thereby predicting said bile flow.

2. The method of claim 1, further comprising calculating
(g) a spatial profile of bile flow velocity and/or pressure in said canaliculi as a function of the coordinate along said axis defined in step (a) and based on the bile transport calculated in step (f); and/or
(h) a second correction factor accounting for a peristaltic component of bile flow.

3. The method of any one of the preceding claims, wherein bile flow through
(a) a single liver lobule;
(b) a plurality of lobules, preferably adjacent lobules; or
(c) an entire liver
is predicted.

4. The method of any one of the preceding claims, wherein
(i) the parameters for steps (b) and (c) are obtained by measurements using (a) intravital microscopy (IVM) movie(s) or movie(s) obtained by non-invasive imaging methods, e.g. Raman microscopy or micro-MRI of one or more detectable bile tracer molecule(s) flowing through the bile canaliculi in vivo, preferably a fluorescent molecule such as 6-carboxyfluorescein-diacetate, said movie preferably being subjected to image analysis;
(ii) the representation of step (d) is obtained from confocal microscopy; and/or
(iii) the data for step (e) is obtained from electron microscopy.

5. The method of claim 4(i), wherein artifacts introduced by motion of the liver or part thereof during said intravital microscopy (IVM) or said non-invasive imaging methods are reduced or removed by embedding said liver or part thereof in vivo in a water-based gel and/or by correcting said IVM movies or movies obtained by non-invasive methods by image processing.

6. The method of any one of claims 2 to 5, wherein the relative magnitude of said peristaltic component is determined by comparing bile flow calculated by said method to bile flow observed experimentally under conditions where osmosis and peristalsis coexist and/or conditions with perturbed acto-myosin contractility, for example upon administration of Fasudil.

7. Use of
(a) a three-dimensional representation of the bile canaliculi in a mammalian liver or a lobule thereof for computer-based prediction of bile flow; and/or
(b) partitioning the axis connecting the central vein of a given lobule and a portal vein of said given lobule of a mammalian liver into a central zone, a middle zone and a portal zone, wherein bile transport in each zone is governed by zone-specific parameters.

8. The method of any one of claims 1 to 6 or the use of claim 7, wherein said ordinary differential equations of (c) are as follows:
$$\begin{matrix}\frac{{dC}_{{c}_{cv}}}{dt} = \frac{{V}_{cv} * {k}_{cleav} * {q}_{cv}}{( {q}_{cv} + {C}_{s} ) * {C}_{s}} + {k}_{{l}_{cv}} * {C}_{{b}_{cv}} - \frac{{k}_{pump} * q {1}_{cv}}{( q {1}_{cv} + {C}_{{c}_{cv} )} * {C}_{{c}_{cv}}} & ( Ia ) \\ \frac{{dC}_{{c}_{md}}}{dt} = \frac{{V}_{md} * {k}_{cleav} * {q}_{md}}{( {q}_{md} + {C}_{s} ) * {C}_{s}} + {k}_{{l}_{md}} * {C}_{{b}_{md}} - \frac{{k}_{pump} * q {1}_{md}}{( q {1}_{md} + {C}_{{c}_{md}} ) * {C}_{{c}_{md}}} & ( Ib ) \\ \frac{{dC}_{{c}_{pv}}}{dt} = \frac{{V}_{pv} * {k}_{cleav} * {q}_{pv}}{( {q}_{pv} + {C}_{s} ) * {C}_{s}} + {k}_{{l}_{pv}} * {C}_{{b}_{pv}} - \frac{{k}_{pump} * q {1}_{pv}}{( q {1}_{pv} + {C}_{{c}_{pv}} ) * {C}_{{c}_{pv}}} & ( Ic )\end{matrix}$$
for the cytoplasmic compartment, and:
$$\begin{matrix} \frac{{dC}_{{b}_{cv}}}{dt} = \frac{{k}_{pump} * q {1}_{cv}}{( q {1}_{cv} + {C}_{{c}_{cv}} ) * {C}_{{c}_{cv}}} - {k}_{{l}_{cv}} * {C}_{{b}_{cv}} - {k}_{{t}_{{cv}_{md}}} * {C}_{{b}_{cv}} & ( IIa ) \\ \frac{{dC}_{{b}_{md}}}{dt} = \frac{{k}_{pump} * q {1}_{md}}{( q {1}_{md} + {C}_{{c}_{md}} ) * {C}_{{c}_{md}}} - {k}_{{l}_{md}} * {C}_{{b}_{md}} + {k}_{{t}_{{cv}_{md}}} * {C}_{{b}_{cv}} - {k}_{{t}_{{md}_{pv}}} * {C}_{{b}_{md}} & ( IIb ) \\ \frac{{dC}_{{b}_{pv}}}{dt} = \frac{{k}_{pump} * q {1}_{pv}}{( q {1}_{pv} + {C}_{{c}_{pv}} ) * {C}_{{c}_{pv}}} - {k}_{{l}_{pv}} * {C}_{{b}_{pv}} + {k}_{{t}_{{md}_{pv}}} * {C}_{{b}_{md}} - {k}_{{t}_{{pv}_{out}}} * {C}_{{b}_{pv}} & ( IIc )\end{matrix}$$
for the bile canaliculi compartment,
wherein:
Cₖᵢ are concentrations of bile or tracer molecule, wherein subscript k indicates the compartment in the lobule, k being either cytoplasmic (c) or bile canaliculi (b), and subscript i indicates the zone in accordance with step (a), i being central (cv), middle (md) or portal (pv);
Vᵢ are volumes of respective zones;
qᵢ are metabolic activity rates within respective zones;
q1ᵢ are bile secretion rates within respective zones;
kₜᵢ are transport rates between respective zones;
kₗᵢ is the rate of leakage of the apical membrane in the respective zones (k_{lcv}, k_{lmd}, kₗₚᵥ); k_{cleav} is the conversion rate of a non-detectable form of said tracer molecule into a detectable form; and kₚᵤₘₚ is the transport rate of bile across the apical plasma membrane of hepatocytes;
and wherein
$$\begin{matrix}{v}_{cv} = \frac{{k}_{{t}_{{cv}_{md}}} * {V}_{cv}}{{A}_{cv}} * L & ( IIIa ) \\ {v}_{md} = \frac{{k}_{{t}_{{md}_{pv}}} * {V}_{md}}{{A}_{md}} * L & ( IIIb ) \\ {v}_{pv} = \frac{{k}_{{t}_{{pv}_{out}}} * {V}_{pv}}{{A}_{pv}} * L & ( IIIc )\end{matrix}$$
wherein:
vᵢ are bile fluid velocity at the interface between zones;
kₜᵢ are transport rates between respective zones;
Vᵢ are volumes of respective zones;
Aᵢ are boundary surfaces of respective zones; and
L is the distance between said central vein and said portal vein.

9. The method of any one of claims 1 to 6 or 8, or the use of claim 7 or 8, to the extent step (f-i) as defined in claim 1 is performed, wherein said spatial profile of (g) is as follows:
$$v ( x ) = \frac{RTc}{2 μ} \sqrt{a ( x ) κ μ} \frac{sinh ( \sqrt{\frac{16 κμ {L}^{2}}{{a ( x )}^{3}}} \frac{x}{L} )}{cosh ( \sqrt{\frac{16 κμ {L}^{2}}{{a ( x )}^{3}}} )} \qquad ( IV )$$
wherein:
v(x) is the bile fluid velocity at a given coordinate x;
x is the coordinate along the axis defined in (a), x being 0 at the central vein and L at the bile duct;
a(x) is hydraulic radius according to step (e) radius at given coordinate x;
R is the universal gas constant;
T is temperature;
μ is the fluid viscosity of bile;
κ is the water permeability of the apical membrane of hepatocytes;
c is a scale factor; and
L is as defined in claim 8.

10. The method or use of claim 9, wherein parameters c and κ in equation (IV) are determined by fitting to velocity at the boundaries of zones as defined by equations (IIIa) to (IIIc) in claim 8.

11. A computer program comprising instructions to cause a computer to execute the steps of the method of any one of claims 1 to 6.

12. A computer-readable medium
(a) comprising instructions which, when executed on a computer, cause said computer to execute the steps of the method of any one of the preceding claims; and/or
(b) having stored thereon the computer program of claim 11.

13. A computer comprising means for carrying out the method of any one of the preceding claims, such means preferably being the computer program of claim 11 and/or the computer-readable medium of claim 12.

14. Use of the method of any one of claims 1 to 6, the computer program of claim 11, the medium of claim 12, or the computer of claim 13
(a) for predicting bile flow upon administration of an agent, lead compound or drug;
(b) for predicting drug-induced liver injury by an agent, lead compound or drug;
(c) in silico safety assessment of an agent, lead compound or drug;
(d) in diagnosis, in particular of cholestatic subtypes;
(e) in personalized medicine; or
(f) for determining the quantitative contribution of peristalsis to bile flow.

15. A method of diagnosing a predisposition for developing cholestasis, liver steatosis, liver fibrosis and/or liver cirrhosis, said method comprising the method of any one of claims 1 to 6, said developing preferably being in response to a disease state or administration of an agent, lead compound or drug.

16. The method of claim 15, wherein said method comprises providing a three-dimensional representation of the bile canaliculi of the patient for whom said predisposition is to be determined.

17. The use of claim 14 or the method of claim 15 or 16, wherein said agent or drug is selected from agents which interfere with the actin cytoskeleton or acto-myosin contractility such as Fasudil; antimicrobials such as isoniazid, rifampin, pyrazinamide, amoxicillin-clavulanate, sulfonamides, nitrofurantoin, minocycline, and ketoconazole; antiretrovirals such as NRTIs, nNRTIs, and protease inhibitors; antiepilectics such as phenytoin, carbamezapine, and valproic acid; analgesics such as NSAIDs including acetoamiphen; lipid lowering agents including statins; immunologics including TNF antagonists; herbal and dietary supplements such as ephedra, green tea extract, and muscle enhancers.
